(19) United States
(12) Patent Application Publication
(10) Pub. No.: US 2021/0046261 A1
(43) Pub. Date: Feb. 18, 2021

(54) METHODS AND SYSTEMS FOR DELIVERING A DOSE USING A VAPORIZER

(71) Applicant: Airgraft Inc., Montreal (CA)

(72) Inventors: Mladen BARBARIC, Westmount (CA); Kisae KIM, Westmount (CA); Sungmoon KIM, Brossard (CA); Bong Geun KIM, Candiac (CA); Chongchun MOON, La Prairie (CA); Nathan Songa YAPI, Montreal (CA); Luca CORBELLINI, Montreal (CA)

(21) Appl. No.: 16/993,165

(22) Filed: Aug. 13, 2020

Related U.S. Application Data
(60) Provisional application No. 62/886,256, filed on Aug. 13, 2019.

(51) Int. Cl. A61M 11/04; A61M 11/00

(52) U.S. Cl. CPC A61M 11/042; A61M 11/005; A61M 2210/0625; A61M 2205/60; A61M 2205/3553

(57) ABSTRACT

In some embodiments, a system includes a mouthpiece defining a mouthpiece opening, a reservoir configured to contain carrier material including one or more constituent substances, a heating assembly including a heating element configured to apply heat to the carrier material to vaporize the carrier material, and a control assembly. The control assembly can determine a dosage of each of the one or more constituent substances delivered to the user based on a volume of the gaseous mixture delivered to the user in response to the vaporization of the portion of the carrier material and based on a data set including an amount of each of the one or more constituent substances included in a standard volume of the gaseous mixture.

### CROSS-REFERENCE TO RELATED APPLICATIONS

This application claims priority to and the benefit of U.S. Provisional Application No. 62/886,256 filed Aug. 13, 2019, entitled “METHODS AND SYSTEMS FOR DELIVERING A DOSE USING A VAPORIZER,” the entire contents of which are hereby expressly incorporated by reference for all purposes.

### BACKGROUND

Electronic vapor delivery systems are increasingly popular. Such systems have been developed for inhalation-based delivery of cannabis components and nicotine. While there is an increasing number of users of electronic vapor delivery systems, users often lack knowledge of the amount of a volatilized substance they inhale when using an electronic vapor delivery system. Users also lack control over the amount or concentration of a volatilized substance they inhale while using electronic vapor delivery systems. Thus, there is a need for methods and systems for monitoring and/or controlling delivery of a dose of a volatile substance such that the volatile substance can be vaporized until a desired dose is reached. There is also a need for methods and systems for ceasing vaporization after a proper and/or desired dose of a volatile substance has been delivered.

### SUMMARY

In some embodiments, a control assembly of a vaporizer device can be configured to determine a set of constituent substances included in a carrier material within a capsule coupled to the vaporizer device. The control assembly can be configured to determine, based on a volume of a gaseous mixture delivered to the user during the vaporization of portion of the carrier material, a dose of a set of constituent substances of the carrier material. During a period of continuous suction on the mouthpiece opening (e.g., a draw), the control assembly can continue to apply current to the heating element (e.g., at intervals) such that the temperature of the heating element remains within a temperature range determined by the control assembly to vaporize the carrier material and an amount of the constituent substances can be drawn from the vaporizer device as aerosols included in the gaseous mixture. The control assembly can be configured to receive a data set including an amount of one or more constituent substances of a set of constituent substances included in a standard volume of a gaseous mixture, the gaseous mixture including air and a volatized portion of the carrier material. Based, at least in part, on the volume of gaseous mixture drawn from the vaporizer and/or delivered to the user following the vaporization of the portion of the carrier material, the control assembly can determine a dose of each of a set of constituent substances delivered to the user as aerosols, the set of constituent substances being included in the carrier material. The control assembly can identify a relationship between the standard volume of the gaseous mixture and the volume of the gaseous mixture delivered to the user during the particular draw. Based on the volume of the gaseous mixture delivered to the user and the amount of the one or more constituent substances of the set of constituent substances included in the standard volume of the gaseous mixture, and the relationship between the standard volume of the gaseous mixture and the volume of the gaseous mixture delivered to the user during the particular draw, the processor can determine a dose of each of the one or more constituent substances delivered to the user during the particular draw. The control assembly can be configured to cease application of the current to the heating element upon receiving an indication of the dose crossing a pre-determined threshold value.

### BRIEF DESCRIPTION OF THE DRAWINGS

FIG. 1 is a schematic block diagram of a system configured for controlled delivery of a dose of substances via a vaporizer, according to an embodiment.

FIG. 2 is a schematic block diagram of a vaporizer, configured for controlled delivery of a dose of substances, according to an embodiment.

FIG. 3 is a schematic block diagram of a compute device included in a controlled dose delivery system, according to an embodiment.

FIG. 4 is a schematic illustration of four charts demonstrating the constituent substances and respective boiling points of the constituent substances of four example carrier materials, according to an embodiment.

FIG. 5A is a plot of a standard volume of a gaseous mixture delivered to a user, shown as a plot of a standard rate of flow as a function of time duration of a standard draw, using a vaporizer, according to an embodiment.

FIG. 5B is an example laboratory report quantifying the measured amounts of each constituent substance of a set of constituent substances included in a standard volume of a gaseous mixture that includes a volatilized portion of an example carrier material.

FIG. 6A is an illustration of an example method of indication of a dose of a carrier material delivered to a user via a vaporizer, according to an embodiment.

FIG. 6B is an illustration of an example method of control of a dose of a carrier material delivered to a user via a user interface in a compute device coupled to the vaporizer of FIG. 6A, according to an embodiment.

FIG. 7 is a plot of an example heating profile of a heating element of a vaporizer during a controlled delivery of a carrier material associated with a single draw applied to the vaporizer, according to an embodiment.

FIG. 8 is a flowchart of an example method of determining a dose of each constituent substance, of a set of constituent substances included in a carrier material, delivered to a user by a vaporizer, according to an embodiment.

FIG. 9 is a flowchart of an example method of controlling a delivery of a dose of a set of constituent substances included in a carrier material via a vaporizer, according to an embodiment.

FIG. 10 is a flowchart of an example method of determining a delivery of a dose of a set of constituent substances included in a carrier material via a vaporizer, according to an embodiment.

FIG. 11 is an illustration of an example method of control of a dose of a carrier material delivered to a user via a user interface in a compute device coupled to a vaporizer, according to an embodiment.

### DETAILED DESCRIPTION

As the popularity of, and commercial interest in, electronic vapor delivery systems (also referred to as “vapor devices” or “vaporizers”) such as electronic cigarettes (“e-cigs”) continues to grow, their manufacture and distribution is becoming more globally widespread. While vaporizers are used to deliver a variety of volatilized substances as aerosols, there is a lack of user feedback about the precise amount of a volatilized substance being delivered during a use of a vaporizer. Furthermore, the amount of a volatilized substance being delivered during an instance of use of a vaporizer (e.g., a draw (also referred to as a puff) that includes a period of continuous suction) can depend on various factors. For example, a strength or intensity of draw (e.g., based on a flow rate through the vaporizer) and a duration of draw can affect a cumulative amount of a volatilized substance delivered. Systems and methods for improved electronic vapor delivery, including control of and user feedback related to a dose of vaporized substances are set forth herein.

In some embodiments, a system includes a mouthpiece defining a mouthpiece opening, a reservoir configured to contain carrier material including one or more constituent substances, a heating assembly including a heating element configured to apply heat to the carrier material to vaporize the carrier material, and a control assembly. The control assembly can supply a current to the heating element in response to an initiation of suction applied to the mouthpiece opening by a user to cause the heating element to vaporize a portion of the carrier material. Additionally, the control assembly can determine a volume of a gaseous mixture delivered to the user in response to the vaporization of the portion of the carrier material, the gaseous mixture including air and a volatized portion of the carrier material. The control assembly can determine a dosage of each of the one or more constituent substances delivered to the user based on the volume of the gaseous mixture delivered to the user in response to the vaporization of the portion of the carrier material and based on a data set including an amount of each of the one or more constituent substances included in a standard volume of the gaseous mixture.

In some embodiments, a method includes supplying a current to a heating element in response to an initiation of suction applied to a mouthpiece opening by a user to cause the heating element to vaporize a portion of a carrier material contained in a reservoir associated with the vaporizer. A volume of a gaseous mixture delivered to the user through the mouthpiece opening in response to the vaporization of the portion of the carrier material can be determined. The gaseous mixture including air and a volatized portion of the carrier material. A dosage of one or more constituent substances of the carrier material delivered to the user based on the volume of the gaseous mixture delivered to the user in response to the vaporization of the portion of the carrier material and based on a data set including an amount of each of the one or more constituent substances included in a standard volume of the gaseous mixture can be determined.

In some embodiments, a method includes receiving data including an identity of a carrier material disposed in a reservoir associated with a vaporizer. A data set including an amount of one or more constituent substances of a set of constituent substances included in a standard volume of a gaseous mixture can be received. The gaseous mixture including air and a volatized portion of the carrier material. Based on a volume of gaseous mixture delivered to the user and the amount of the one or more constituent substances of the set of constituent substances included in the standard volume of the gaseous mixture, a dosage of each of the one or more constituent substances delivered to the user can be determined.

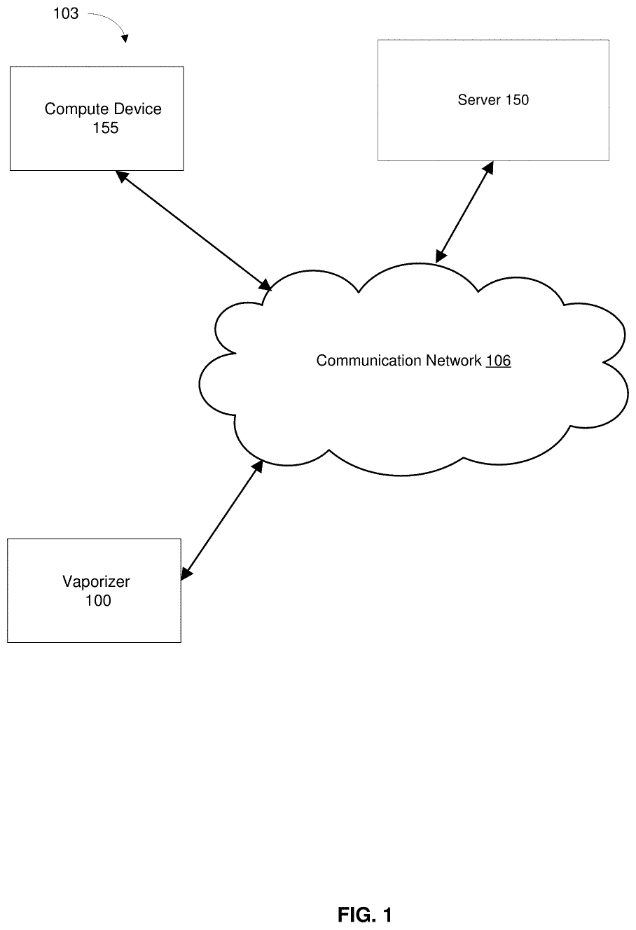

FIG. 1A is a schematic block diagram of a system 103 for controlled delivery of a volatilized substance (e.g., carrier material) including a set of constituent substances. The system 103 is configured to provide user control and feedback related to a delivery of a dose of the volatilized substance. The system 103 can determine (e.g., calculate or identify) a dose of each of one or more constituent substances included in the volatilized substance that is delivered to a user of a vaporizer during an instance of usage. In some embodiments, the system 103 can be configured such that a user can control the dose of one or more of the constituent substances included in the carrier oil. In some embodiments, the system 103 can educate the user, based, for example, on a determined dose of one or more of the constituent substances included in the volatized substance, to maintain and/or alter one or more parameters associated with their usage of the vaporizer (such as, for example, draw length, draw intensity, frequency of use, etc.) to achieve an intended dose.

The system 103 includes a vaporizer 100, a compute device 155, and a server 150 connected to one another via a communication network 106, as illustrated in FIG. 1. While the system 103 is illustrated as including one vaporizer 100, one compute device 155 and one server 150, in some embodiments the system 103 can include any suitable number of vaporizers, compute devices, and/or servers.

In some embodiments, the communication network 106 (also referred to as “the network”) can be any suitable communication network for transferring data and can operate over public and/or private networks. For example, the network 106 can include a private network, a Virtual Private Network (VPN), a Multiprotocol Label Switching (MPLS) circuit, the Internet, an intranet, a local area network (LAN), a wide area network (WAN), a metropolitan area network (MAN), a worldwide interoperability for microwave access network (WiMAX®), an optical fiber (or fiber optic)-based network, a Bluetooth® network, a virtual network, and/or any combination thereof. In some instances, the communication network 106 can be a wireless network such as, for example, a Wi-Fi or wireless local area network (“WLAN”), a wireless wide area network (“WWAN”), and/or a cellular network. In other instances, the communication network 106 can be a wired network such as, for example, an Ethernet network, a digital subscription line (“DSL”) network, a broadband network, and/or a fiber-optic network. In some instances, the network can use Application Programming Interfaces (APIs) and/or data interchange formats, (e.g., Representational State Transfer (REST), JavaScript Object Notation (JSON), Extensible Markup Language (XML), Simple Object Access Protocol (SOAP), and/or Java Message Service (JMS)). The communications sent via the network 106 can be encrypted or unencrypted. In some instances, the communication network 106 can include multiple networks or subnetworks operatively coupled to one another by, for example, network bridges, routers, switches, gateways and/or the like (not shown). In some embodiments, the compute device 155 can communicate directly with the vaporizer 100.

The server 150 can be associated with one or more processors and/or memory (not shown). In some embodiments, the server 150 can be a remote database server that is configured to receive signals and send information or data sets to one or more vaporizers and/or compute devices. In some embodiments, the server 150 can be hosted by one or more third parties (e.g., laboratories, manufacturers, vendors, licensing institutions, certified institutions moderating standardized practices for the usage of vaporizers, etc.). The server 150 can be configured to send, in response to a query based on an identifier, data associated with an identity of a carrier material related to the identifier. In some embodiments, the server 150 can be configured to provide information associated with standard gaseous mixtures. For example, the server 150 can be configured to provide an amount of one or more constituent substances included in a standard volume of a gaseous mixture, the gaseous mixture including air and a volatized portion of a particular carrier material associated with an identifier. In some embodiments, the server 150 can be configured to provide data associated with standard use of one or more vaporizers based on a set of parameters such as make or model of the vaporizer, geographical region of use, etc.

In some embodiments, the carrier material can include a set of constituent substances (e.g., compounds) included in the carrier material (including one or more additives if applicable). The carrier material, like any of the carrier materials described herein, can be and/or include any suitable material (e.g., an oil) configured to be vaporized and inhaled by a user. For example, the carrier material can include cannabis, nicotine, propylene glycol, plant-based oils, and/or pharmaceuticals configured to be vaporized for inhalation. Specifically, in some embodiments, the carrier material can include any number of substances including phytocannabinoids, terpenes, and/or other substances that can be volatilized.

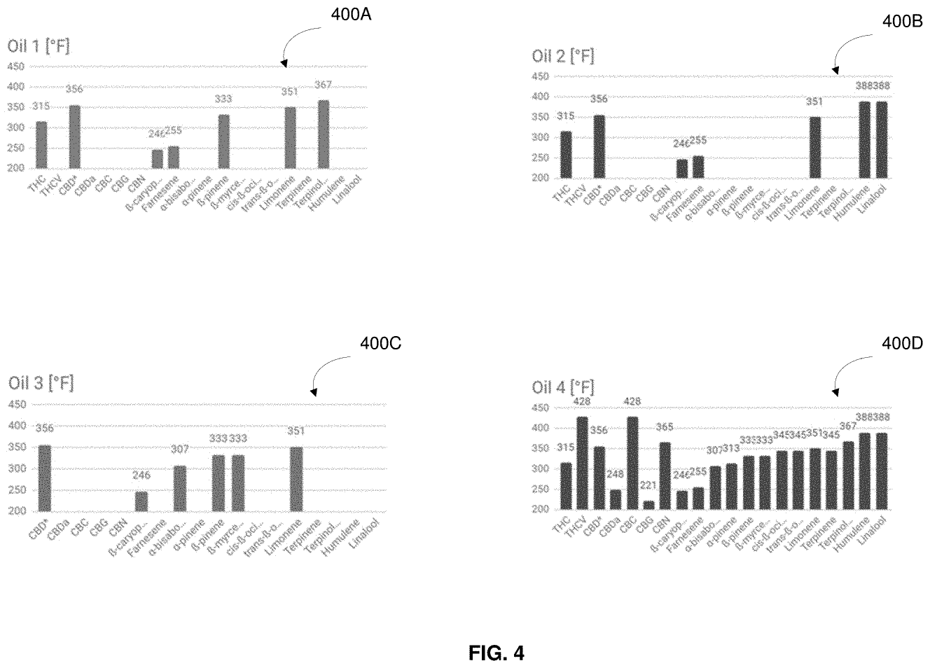

The carrier material can include different constituent substances (e.g., compounds included in liquid mediums such as plant-based oils) having different boiling temperatures, combustion temperatures, temperature sensitivities, or temperature ranges associated with different drug efficacy levels. Some example phytocannabinoids that can be included in a carrier material (e.g., oil) include tetrahydrocannabinol (THC), Delta-8 tetrahydrocannabinol (Δ-8-THC), tetrahydrocannabivarin (THCV), cannabidiol (CBD), Cannabidivarin (CBDV), Cannabinol (CBN), Cannabichromene (CBC), Cannabigerol (CBG), their acidic precursors, such as tetrahydrocannabinolic acid (THCA), cannabidiolic acid (CBDA), and cannabigerolic acid (CBGA), and/or their derivatives. Some example terpenes that can be included in a carrier material (e.g., oil) include citronellol, d-limonene, famesene, humulene, linalool, β-caryophyllene, β-myrcene, β-pinene, and terpineol-4-ol. FIG. 4 illustrates four charts 400A-D demonstrating the constituent substances and respective boiling points of the constituent substances of four example carrier materials (referred to as “Oil 1,” “Oil 2,” “Oil 3,” and “Oil 4”). Each chart represents a boiling point profile of each carrier material that is represented as a bar chart showing the comparative boiling points of each constituent substance of each carrier material. As shown, Oil 3 includes the fewest constituent substances and Oil 4 includes the most constituent substances, each constituent substance having a different boiling point. A first carrier material (e.g., Oil 3) may include a first set of constituent substances which when vaporized can deliver a first dose of the first set of constituent substances to the user and a second carrier material (e.g., Oil 4) may include a second set of constituent substances different from the first set, which when vaporized can deliver a second dose of the second set of constituent substances to the user. Carrier materials having different compositions of constituent substances can have different doses included in a vaporized portion of the carrier material (e.g., within a volume of a gaseous mixture including the vaporized portion of the carrier material). Additionally, carrier materials heated and/or vaporized according to different heating profiles can have different doses included in a vaporized portion of the carrier material (e.g., within a volume of a gaseous mixture including the vaporized portion of the carrier material) due to some constituent substances boiling at a lower temperature than other constituent sub stances.

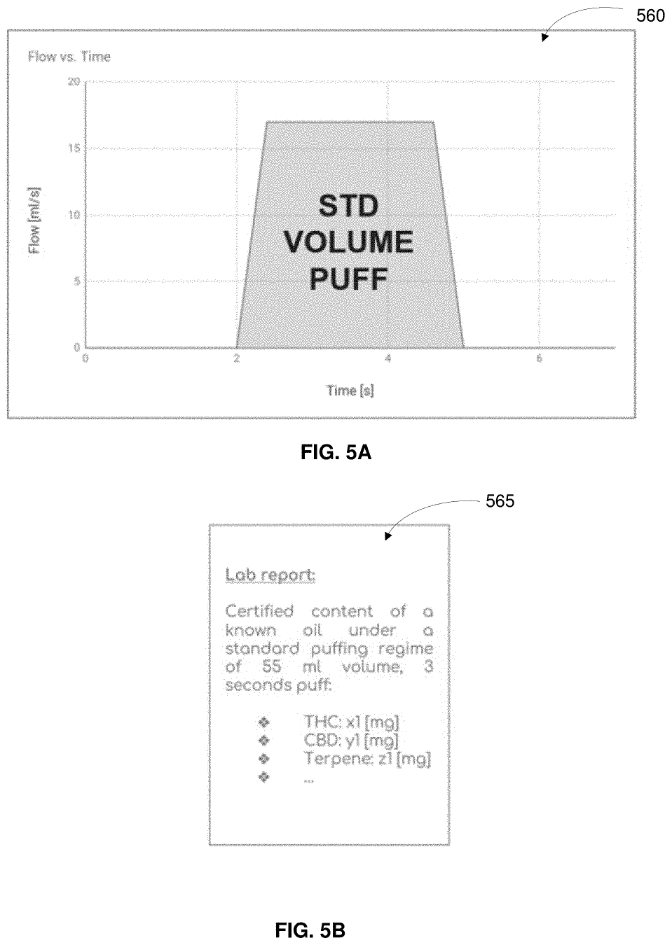

In some embodiments, the server 150 can be configured to provide data associated with a carrier material disposed in the vaporizer 100 such as a set of constituent substances, relative amounts of the constituent substances included in the carrier material, and/or boiling points of the constituent substances included in the carrier material. For example, the server 150 can include a set of constituent substances, relative amounts of the constituent substances, and/or boiling points of the constituent substances included in a standard volume of the carrier material. FIG. 5A illustrates an example representation 560 of a standard volume of a gaseous mixture plotted as a quantification of flow rate as a function of time (e.g., delivered to a user or drawn by a user by vaporizing a known carrier material under a standard regime of usage of a vaporizer, such as a vaporizer tested using a Routine Analytical Machine for E-Cigarette Aerosol generation and collection as recommended by a task force associated with the Cooperation Center for Scientific Research Relative to Tobacco (CORESTA)). The standard volume of a gaseous mixture can be a volume of gaseous mixture associated with a standard draw or a standard puff (e.g., a standard duration of suction at a rate above a threshold flow rate resulting in a decrease in pressure within the vaporizer below a threshold pressure applied to a mouthpiece opening). The standard volume can be obtained from an external regulatory body or organization (e.g., from studies conducted by the task force associated with CORESTA). The standard volume (e.g., based on the findings of the CORESTA Task Force) can be defined as a 55 ml puff volume. Additionally, the standard puff can be defined as having a 3 second puff duration. Furthermore, in some embodiments, the standard puff can be associated with a 30 second puff interval (between two consecutive puffs) and/or a square wave puff profile. In some embodiments, the standard volume can have any suitable volume, such as, for example, 55 ml, 65 ml, 75 ml, 85 ml, 95 ml, 105 ml, or any value in between. In some embodiments, the standard duration of a puff can be any suitable duration, such as, for example, 3 seconds, 4 seconds, 5 seconds, 6 second, 7 seconds, or any value in between. In some embodiments, the puff interval can be any suitable puff interval, such as, for example, 5, seconds, 10 seconds, 15 seconds, 20 seconds, 25 seconds, 30 seconds, or any value in between.

FIG. 5B illustrates an example laboratory report 565 quantifying a certified standard amount of the one or more constituent substances included in the known carrier material delivered during the draw of the standard volume of the gaseous mixture (e.g., the volume of a standard puff) generated from vaporizing the known carrier material under the standard regime of usage of the vaporizer (e.g. x1 mg of Tetrahydrocannabinol (THC), y1 mg of Cannabidiol (CBD), z1 mg of terpene, in a 55 ml standard volume associated with a standard draw with a duration of 3 seconds).

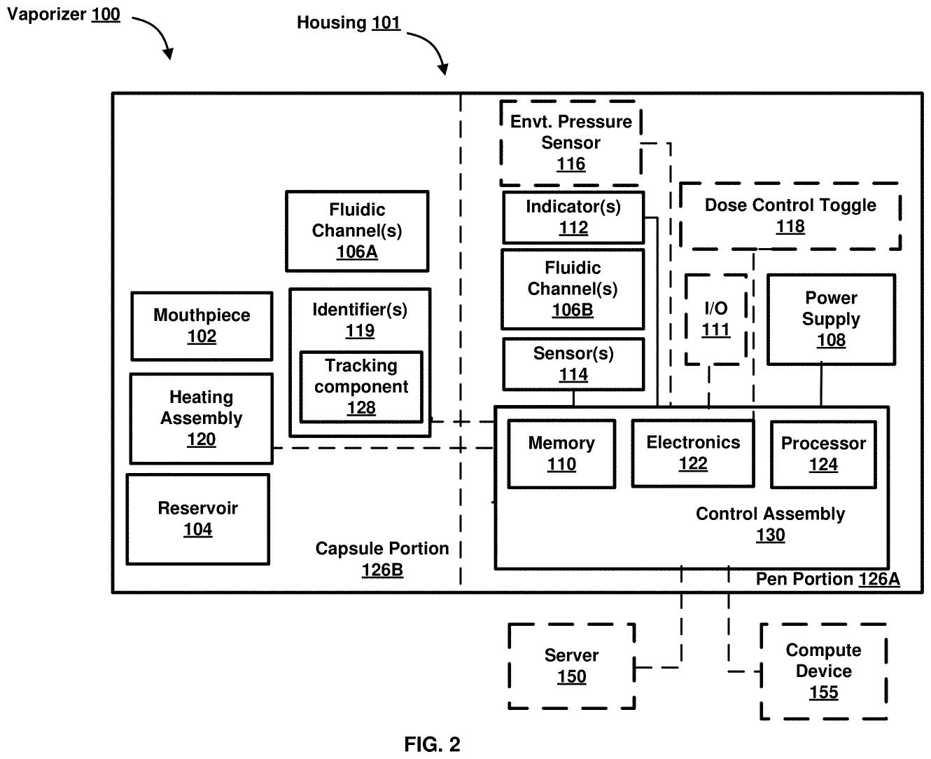

The vaporizer 100 can be a vaporizing device used to consume inhaled volatile substances using a controlled dose delivery and/or for providing feedback of a delivered dose of the volatile substances, according to an embodiment. The vaporizer 100 can be configured to communicate with the compute device 155 and/or the server 150 via the communication network 106, as shown in FIG. 1. In some embodiments, the vaporizer 100 can be configured to communicate with the server 150 to send/receive one or more data sets associated with one or more carrier materials disposed within the vaporizer 100 and/or associated with characteristics or use of the vaporizer 100, as described in further detail herein. In some embodiments, the vaporizer 100 can be operatively coupled to the compute device 155 (e.g., via the communication network 106) such that a user can be provided feedback and/or control of a dose of one or more constituent substances included in a carrier material used in the vaporizer 100, as described in further detail herein. In some embodiments, the vaporizer 100 can be operatively coupled to the server 150 via the compute device 155 such that the compute device 155 can provide a heating profile associated with a carrier material and/or doses of constituent substances in a carrier material of the vaporizer 100 to the vaporizer 100 from the compute device 155. FIG. 2 illustrates a schematic block diagram of the vaporizer 100 shown in the system 103 of FIG. 1. The vaporizer 100 can be configured for controlled delivery of a dose of substances, according to an embodiment.

In some embodiments, the system 103 in FIG. 1 and/or the vaporizer 100 in FIGS. 1 and 2 can be substantially the same or similar in structure and/or function to any of the systems or vaporizers described in the U.S. patent application Ser. No. 16/655,153, filed on Oct. 16, 2019, published as US2020/0113246, entitled “Variable-Viscosity Carrier Vaporizers with Enhanced Thermal and Hydrodynamic Properties” which is incorporated by reference herein in its entirety, for all purposes.

As shown in FIG. 2, the vaporizer 100 includes a housing 101 that includes a pen portion 126A and a capsule portion 126B (also referred to herein as a “capsule”). The capsule portion 126B includes a mouthpiece 102, a reservoir 104, one or more fluidic channels 106A (e.g., microfluidics or other passageways), a heating assembly 120, and one or more identifiers 119. The identifiers 119 can optionally include a tracking component 128. The pen portion 126A includes one or more fluidic channels 106B (e.g., microfluidics or other passageways), one or more of a power supply 108, an optional input/output (I/O) module 111, one or more sensors 114, indicator(s) 112, and a control assembly 130. The one or more sensors 114 can include, for example, a pressure sensor. The control assembly 130 can include a memory 110, electronics 122, and a processor 124 (e.g., coupled to a printed circuit board). The electronics 122 can include communication electronics via which the vaporizer 100 can be configured to communicate with the server 150 and/or the compute device 155 system 103. In some embodiments, the vaporizer 100 can optionally include a dose control toggle 118 (e.g., a button) configured to set a threshold associated with a dose of one or more constituent substances of a carrier material. The vaporizer 100 can optionally include, in the pen portion 126A, an environmental pressure sensor 116, according to some embodiments.

The mouthpiece 102 can comprise one or more of ceramic, heat-resistant plastic, anodized aluminum, or any other suitable material. The power supply 108 can include any suitable battery or fuel cell, for example having high-drain characteristics. The reservoir 104 can include a carrier material and can be in fluid communication with at least one of the mouthpiece 102, the one or more chambers (e.g., vapor expansion chambers, not shown), and the fluidic channel(s) 106A and 106B, such that carrier material can travel from the reservoir 104 into a fluid path (also referred to as an air flow path) defined by the mouthpiece 102, the one or more chambers, and the fluidic channel(s) 106A and 106B as a result of triggering heating and vaporization of the carrier material. In some embodiments, heating of the carrier material can be initiated by the control assembly 130 in response to a user's sucking/drawing on the mouthpiece 102 during use (e.g., via activation of a flow sensor of the sensor(s) 114). In some embodiments, the vaporizer 100 can include a mechanical interface (e.g., a button) (e.g., included in the input/output module 111) that the user can manually actuate to trigger the heating and vaporization of the carrier material.

The memory 110 can include any electronic component capable of storing electronic information. The term memory may refer to various types of processor-readable media such as random access memory (RAM), read-only memory (ROM), non-volatile random access memory (NVRAM), programmable read-only memory (PROM), erasable programmable read only memory (EPROM), electrically erasable PROM (EEPROM), flash memory, magnetic or optical data storage, registers, etc. Memory is said to be in electronic communication with a processor if the processor can read information from and/or write information to the memory. Memory that is integral to a processor is in electronic communication with the processor.

The processor 124 can include one or more of: a general purpose processor, a central processing unit (CPU), a microprocessor, a digital signal processor (DSP), a controller, a microcontroller, a state machine and so forth. Under some circumstances, a “processor” may refer to an application specific integrated circuit (ASIC), a programmable logic device (PLD), a field programmable gate array (FPGA), etc. The term “processor” may refer to a combination of processing devices, e.g., a combination of a DSP and a microprocessor, a plurality of microprocessors, one or more microprocessors in conjunction with a DSP core or any other such configuration.

In some embodiments, the vaporizer 100 can be a reusable vaporizer, such that the pen portion 126A can be reused and the capsule portion 126 can be renewed for each use. In such embodiments, to assemble the vaporizer 100, a user may, prior to use (e.g., upon purchase of a new capsule), connect the capsule 126B with the pen portion 126A of the vaporizer 100. The capsule 126B and the pen portion 126A can be configured to be mechanically and electrically connected, for example by one or more of screw attachment, press-fit attachment, snap-fit attachment, magnetic attachment, or any other suitable connection means. As can be inferred from the foregoing, the pen portion 126A can be considered to be a reusable portion of the vaporizer 100B, and the capsule 126B can be considered to be a disposable or “replaceable” portion of the vaporizer 100B For example, the control assembly 130 can be configured to be coupled to the tracking component 128 when the capsule 126B is coupled to the pen portion 126A such that the control assembly 130 can access information contained in the tracking component 128. The tracking component 128 may be, for example, an integrated circuit (e.g., Application-Specific Integrated Circuits (ASICs)). The tracking component 128 can be configured to contain data related to the capsule 126B. In some implementations, the tracking component 128 may contain capsule identification information corresponding to the capsule 126B such that the control assembly 130 may recognize the capsule 126B and such that information about the contents of the capsule 126B can be received from the tracking component 128 by the processor 124. In some embodiments, the vaporizer 200 can be a disposable (or “single-use”) vaporizer. The tracking component 128 may contain information related to the specific carrier material disposed in the reservoir 106. In some implementations, the tracking component 128 may provide information related to an identity of the specific carrier material or specifying one or more constituent substances included in the carrier material. The information from the tracking component 128 may be used to calculate a dose of each of the one or more constituent substances included in the carrier material being delivered to a user during an instance of usage of the vaporizer, by appropriately vaporizing the carrier material via applying a particular current and/or voltage to the heating element for a suitable amount of time. In some implementations, the tracking component 128 can store a heating profile associated with the specific carrier material within the reservoir 106 and can provide the heating profile to the control assembly 130 of the pen portion 126A. In some implementations, the tracking component 128 can store a dosage information of one or more constituent substances included in a standard volume of the carrier material (e.g., when included in a gaseous mixture vaporized at a particular vaporization temperature) and can provide the dosage information of the one or more constituent substances included in the standard volume to the control assembly 130 of the pen portion 126A.

The input/output module 111 can include one or more of: a push-button control for causing vapor generation, a battery indicator, an electromechanical connector for charging and/or data communication, a light source (e.g., one or more light-emitting diodes), etc. The heating assembly 120 can include a heating element such as, for example, a coil heater, a rod-shaped heater, a pancake heater, a chemical heater, or any other heater that is sized, dimensioned, and constituted of material suitable for heating a carrier material. In some embodiments, for example, the heating assembly can include a ceramic cylindrical wick portion defining a central passageway, a coil coupled to and/or disposed within the cylindrical wick portion configured to heat the cylindrical wick portion, and a cotton wick portion wrapped around the outer surface of the cylindrical wick portion. In some embodiments, for example, the heating assembly can include a wick (e.g., a cotton wick) and a coil having a portion wrapped around the wick and two ends extending away from the wick. The two ends can be configured to be coupled to the control assembly 130 such that the temperature of the coil can be controlled, at least in part, by a current applied to the ends of the coil. The wick can be configured to transport carrier material toward a portion of the wick adjacent the coil.

The electronics 122 can include any suitable communication component configured to allow communication of information (e.g., data) from a remote compute device 155 and/or a remote server 150 to the control assembly 130 (e.g., to the processor 124). For example, the electronics 122 can include one or more of: a GPS receiver, an antenna, heater control circuitry (e.g., configured to control a temperature of the heating element of the heating assembly 120), or a transceiver for wireless (e.g., Bluetooth®) communication with a command center or other remote compute device (such as a mobile device of a user). The sensor(s) 114 can include one or more of: a flow sensor, a pressure sensor, a temperature sensor, a position sensor, an orientation sensor, etc. In some embodiments, a pressure sensor included in the sensor(s) 114 can be configured to measure a change in pressure within the vaporizer 100 (e.g., a change in pressure within one of the fluidic channels 106B in fluid communication with the fluid path that includes the fluidic channel(s) 106A, the fluidic channel(s) 106B, the one or more chambers, and the mouthpiece opening of the vaporizer 100). The optional environmental pressure sensor 116 can be configured to determine (e.g., measure) an environmental pressure such that the processor 124 can account for potential changes in vapor pressure and/or boiling points of the substances included in the carrier material arising from environmental pressure (e.g., at high altitude regions) to appropriately vaporize the substances, to determine (e.g., calculate) a dose of one or more of the substances, and/or to control the dose of the one or more substances during use of the vaporizer 100. The identifier(s) 119 may include, for example, a bar code, a QR code, and/or a near-field communication (NFC) device such that the vaporizer 100 may be identified and/or recognized by an external device (e.g., the compute device 155). The indicator(s) 112 can include one or more of: an illumination source (e.g., one or more light-emitting diodes), a speaker, a display screen, haptic feedback component (e.g., a vibration element or motor), etc. For example, in some embodiments, the indicator(s) 112 may include indicator features. The indicator features may include, for example, light-emitting diodes (LEDs). The indicator features may be configured to transmit light through a set of apertures in the housing of the vaporizer. The indicator features may be configured to indicate via one or more parameters associated with the light emitted (e.g., a wavelength of light emitted via the indicator features) such as a dose of one or more of the constituent substances included in the carrier material, a strength of a draw, and/or an indication of a relative degree of volume of gaseous mixture inhaled during a particular draw using the vaporizer 100 (e.g., an indication of how much of a portion of an intended draw or an amount of a substance included in the carrier material has been drawn from the mouthpiece 102).

In some embodiments, in use, the vaporizer 100 can be configured such that, when a user sucks, or “draws,” on an opening defined by the mouthpiece 102, the resulting change in pressure within the vaporizer 100 is measured by a sensor 114. In response to the sensor 114 sensing a change in pressure (e.g., above a threshold change in pressure or to a threshold pressure level), the processor 124 can actuate the heater control circuitry of the electronics 122 to pass a current through the heating element that is in contact with, or in sufficiently close proximity to, the carrier material or a wick material containing at least a portion of the carrier material, so as to cause the volatilization of a portion of the carrier material. The volatilized carrier material, or vapor, travels toward the mouthpiece via one or more of the expansion chamber(s) and exits the vaporizer via the opening in the mouthpiece for inhalation by the user.

In some embodiments, the vaporizer 100 can provide a user feedback related to a dose of one or more constituent substances of a carrier material delivered during a use of the vaporizer (e.g., during a draw of the vaporizer). In some embodiments, the feedback can be provided via the indicator(s) 112 and/or via a vaporizer controller application and a user interface displayed on a display associated with the compute device 150, as described in further detail below.

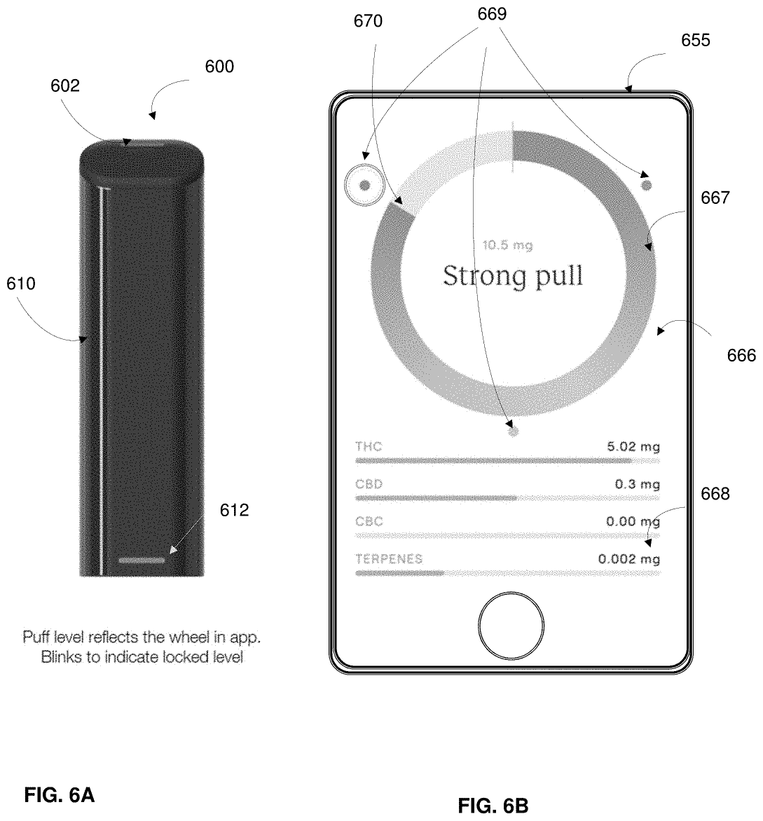

In some embodiments, the control assembly 130 can be configured to control indicator(s) 112 to reflect various states or conditions of the vaporizer 100 during an instance of use. For example, the control assembly 130 can be configured to control the indicator(s) 112 to reflect a dose associated with an instance of use of the vaporizer 100. In some embodiments, the vaporizer 100 may include any suitable number of indicator features associated with the indicator(s) 112, such as, for example, an array of LEDs (also referred to as an LED array). In some implementations, the indicator features may be operatively coupled to the control assembly 130 such that one or more of the indicator features illuminates such that a user can visually identify the dose drawn from the vaporizer (e.g., during and/or after the draw). As an example, in some implementations, the control assembly 130 can be configured to control the indicators 112 to indicate a dose associated with one or more constituent substances of a carrier material and/or a strength of a draw (the strength of a draw corresponding to a flow rate associated with the draw), as shown and described in further detail below, with reference to the embodiment 600 shown in FIG. 6A.

In some implementations, the indicator(s) 112 can be controlled to indicate a degree of volume of gaseous mixture inhaled during a draw, the degree of volume being mapped to a predetermined look-up-table (LUT). As an example, in some implementations, the control assembly 130 can map the dose of one or more constituent substances delivered in a draw or a degree of volume of a draw to a LUT by generating the LUT as an array of varying wavelengths of light across a color map, such that different levels of dose or different strength of draw or different degrees of volume correspond to a different portion of the LUT, respectively. In some implementations, the control assembly 130 can be configured to cause the indicator(s) 112 to indicate a dose or degree of draw by emitting a light of a suitably modulated wavelength that corresponds to the dose or degree of draw according to the mapping of the LUT. For example, a first indication (e.g., a blue wavelength) can be mapped to indicate a low dose, a weak draw, or a relatively small volume compared to a standard dose, a strength of a standard draw, or a standard volume of a gaseous mixture, respectively, during and/or after use of a vaporizer (e.g., during and/or after a user draws fluid through the mouthpiece 102). A second indication (e.g., an orange-red wavelength) can be mapped to indicate a large dose, a strong draw, or a relatively large volume compared to a standard dose, a strength of a standard draw, or a standard volume of a gaseous mixture, respectively, during use of a vaporizer. An example implementation of a control of indicators to indicate a mapped dose or draw volume associated with an instance of use of a vaporizer is described in further detail with reference to FIGS. 6A and 6B below.

During a portion of a ramp up period (e.g., a pre-heating period) in which the temperature of the heating element is increasing but some or all of the constituents of the carrier material may not be evaporating, dosing of those constituents has not yet started. In some embodiments, the volume of a draw or degree of volume of a draw can be measured or calculated starting when the heating element and/or a portion of the carrier material disposed near the heating element reaches a threshold temperature. For example, in some embodiments, the control assembly 130 can determine the dose of the vaporized carrier material included in a draw based, in part, on a time at which a temperature of the heating element and/or of a portion of the carrier material disposed near the heating element crossed a threshold temperature during a ramp up period (e.g., a pre-heating period) or a portion of the ramp up period of the temperature of the heating element and/or the portion of the carrier material disposed near the heating element. In some embodiments, the calculation of the volume of the gaseous mixture drawn through and/or from the vaporizer 100 may be based on a calculation or measurement of the amount of gaseous mixture drawn through and/or from the vaporizer 100 after the heating element and/or the portion of the carrier material disposed near the heating element has reached a threshold percentage of the intended vaporization temperature or temperature range, such as, for example, 50%, 60%, 70%, 80%, or 90%. Similarly, the control assembly 130 can determine the dose of the vaporized carrier material included in a draw based, in part, on a time at which a temperature of the heating element and/or of a portion of the carrier material disposed near the heating element crossed a threshold temperature during a ramp down period (e.g., a cooling period) or a portion of the ramp down period of the temperature of the heating element and/or the portion of the carrier material disposed near the heating element. Thus, any residual vaporization that is expected during an initial portion of the ramp down following the ceasing of application of current can be included in the dose determination.

In some embodiments, the vaporizer 100 can be operatively coupled to the compute device 155 and can send signals, via communication electronics included in the electronics 122, to the compute device 155 to display one or more indications via a user interface instantiated at the compute device 155 by a vaporizer controller application. In such embodiments, in some instances, an indication of different levels of a dose of one or more constituent substances and/or an indication of different strengths of a draw and/or different degrees or amounts of volume inhaled via the indicators 112, as described above, can be synchronized with one or more indications via the display at the compute device 155. In some implementations, as an example, the user interface instantiated at the compute device 155 can include a graphical presentation, for example a wheel, representing a range of values. In some implementations, the range of values can correspond to a range of doses. In some implementations, the range of values can correspond to a range of draw volumes. The wheel can include an indication that is configured to move relative to a first point on the wheel and along the range of values during a draw of the vaporizer 100. The movement of the indication can be programmatically coupled to the draw such that the position of the indication at a given time during a draw is configured to indicate the cumulative dose of a substance drawn by the user, or a cumulative volume of gaseous mixture drawn by the user after a particular time (e.g., after pre-heating or after a particular percentage of pre-heating), at that time of or point in the draw. In some embodiments, a rate of movement of the indication at a point in time during a draw can indicate a flow rate associated with the draw (e.g., a draw strength) at that point in time. The user interface, in some implementations, can include separate dose indicators identifying the particular amount of each substance drawn from the mouthpiece opening. An example implementation of synchronized indications via indicators on a vaporizer and via a display of a compute device coupled to the vaporizer is described in further detail with reference to FIGS. 6A and 6B below.

In some embodiments, a dose of one or more constituent substances drawn from the vaporizer 100 can be controlled and/or limited to a threshold dose. For example, in some implementations, the control assembly 130 can be configured to calculate a dose of one or more constituents based on a volume of gaseous mixture drawn by a user during an instance of use of the vaporizer 100, and an amount of the one or more constituents included in a standard volume of the gaseous mixture. The control assembly 130 can be configured to receive a data set including an amount of one or more constituent substances of a set of constituent substances included in a standard volume of a gaseous mixture, the gaseous mixture including air and a volatized portion of the carrier material. For example, the vaporizer 100 may receive via the server 150 a data set including a standard amount (e.g., a certified standard amount) of the one or more constituent substances included in a known carrier material delivered during a draw of a standard volume of a gaseous mixture generated from vaporizing the known carrier material under a standard regime of usage of a vaporizer (e.g., a standard draw using a standard vaporizer).

As an example, the control assembly 130 can receive, from a pressure sensor of the sensors 114, information related to a change in pressure associated with fluids (e.g., air, gaseous mixture including aerosols from volatilized potion of the carrier material, etc.,) held in one or more portions of the vaporizer 100 (e.g., the fluidic channels 106B) that are in fluid communication with the air flow path through the vaporizer 100 and out of the opening of the mouthpiece 102. The change in pressure may be associated with a change in flow and/or a flow rate of air and gaseous mixture (including air and volatilized portion of the carrier material) through the vaporizer 100 due to suction applied by a user of the vaporizer 100. The control assembly 130 can calculate a volume of the gaseous mixture (e.g., air and a volatized portion of the carrier material) delivered to the user during a particular draw. The control assembly 130 can use the change in pressure data over time and a time duration of the change or fluctuation to determine (e.g., calculate) the flow rate through the vaporizer 100 (e.g., the fluid channels 106B, 106A and chambers) and out of the opening in the mouthpiece 102. The flow rate can then be used to calculate the volume drawn from the vaporizer 100.

As another example, the sensor(s) 114 can include a flow sensor. The control assembly 130 can receive, from the flow sensor, information related to a duration of flow and flow rate of air over a set of time points of gaseous mixture drawn by a user of the vaporizer 100, and the control assembly 130 can calculate, based on the flow rate at each of the set of time points and the duration of the draw, a volume of the gaseous mixture delivered to the user during the particular draw, the gaseous mixture including air and a volatized portion of the carrier material.

The control assembly 130 can identify a relationship (e.g., a ratio) between the standard volume of the gaseous mixture and the volume of the gaseous mixture delivered to the user during the particular draw. Based on the volume of the gaseous mixture delivered to the user and the amount of the one or more constituent substances of the set of constituent substances included in the standard volume of the gaseous mixture, and the relationship between the standard volume of the gaseous mixture and the volume of the gaseous mixture delivered to the user during the particular draw, the dose of the one or more constituents included in the carrier material can be calculated. The control assembly 130 can determine a dose of each of the one or more constituent substances delivered to the user during the particular draw. In some implementations, the dose of each of the one or more constituent substances drawn from the vaporizer 100 can be presented to the user (e.g., via indicator(s) 112 or a user interface of compute device 155).

The control assembly 130 can also receive one or more threshold values associated with as desired dose of the one or more constituent substances. In some implementations, a threshold associated with the dose of one or more of the constituent substances included in the carrier material can be set (e.g., by a user or by a manufacturer of the vaporizer 100 or the carrier material). Based on at least one of the determined dose of the one or more constituent substances, at a given time during a draw, the control assembly 130 can cease delivery of the volatilized constituents into the air flow path of the vaporizer 100 when the dose of one or more of the constituent substances is near, at, or above the threshold. The control assembly 130 can cease continued delivery of vaporized substances by ceasing the supply of current to the heating coil associated with the vaporizer 100, as described in further detail below.

In some embodiments, the vaporizer 100 can be configured such that the user can set the threshold using the optional dose control button 118. In some embodiments, the vaporizer 100 can be configured such that the user can set the threshold via a user interface associated with a vaporizer controller application executed on the compute device 155 operatively coupled to the vaporizer 100, for example, via a touch screen. In some instances, the compute device 155 can include a user interface that provides one or more control items and one or more display items configured to perform functions associated with communication with the vaporizer 100, remotely setting a threshold associated with a dose of substances included in a carrier material delivered during use of e vaporizer 100, and/or display feedback related to a delivered and/or predicted dose of one or more substances included in a carrier material associated with a usage of the vaporizer 100 as described in further detail below.

The vaporizer 100 can be configured to provide to the user the calculated dose as feedback such that the user may either maintain or change one or more parameters of their use of the vaporizer (e.g., use stronger or weaker draws to obtain a larger or smaller dose per use, increase or decrease a frequency of use to obtain a larger or smaller dose over two or more uses over time, etc.). In some embodiments, the vaporizer 100 can be configured to calculate a predicted dose of the one or more constituent substances included in a carrier material given the identity of the carrier material to be used.

In some embodiments, for example, a dose of one or more constituent substances can be controlled via receiving data (e.g., from the capsule portion 126B of the vaporizer 100), the data including an identity of a carrier material included in the reservoir 104 of the capsule portion 126B. Based on the identity, the control assembly 130 can determine the contents of the carrier material, including one or more specific constituent substances included in the carrier material, along with particular characteristics of the constituent substances (e.g., a boiling point and/or combustion point of each constituent substance). Based at least in part on the characteristics of the contents of the carrier material, the control assembly 130 can determine a particular temperature or temperature range at which the carrier material is to be vaporized such that a desired dose of a constituent or a dose less than a threshold value of the constituent in the carrier material is expected to be drawn by a user. In some implementations, the control assembly 130 can determine a dose of the vaporized carrier material drawn from the device based in part on the temperature at which the carrier material is vaporized. For example, the control assembly 130 may determine (e.g., calculate) that a standard volume of a gaseous mixture including a carrier material vaporized at a first vaporization temperature would include a first dose of a first constituent substance and a first dose of a second constituent substance. The control assembly 130 may determine (e.g., calculate) that a standard volume of a gaseous mixture including the carrier material vaporized at a second vaporization temperature higher than the first vaporization temperature would include a second dose of the first constituent substance and a second dose of the second constituent substance. The first dose of the first constituent substance can be non-zero and the first dose of the second constituent substance can be zero if the first vaporization temperature is a temperature below the boiling point of the second constituent substance and above the boiling point of the first constituent substance. The second dose of the first constituent substance and the second dose of the second constituent substance can both be non-zero if the second vaporization temperature is a temperature above the boiling point of the first constituent substance and the second constituent substance. In some embodiments, rather than the control assembly 130 determining the dose of the vaporized carrier material drawn from the device based on the vaporization temperature, the control assembly 130 can receive the dose of a standard volume of a gaseous mixture including a carrier material and receive a particular heating profile associated with the dose of the standard volume. The control assembly 130 can then be configured to implement the particular heating profile and calculate the dose based on the dose of the standard volume of the gaseous mixture and the volume of air drawn from the vaporizer.

As described previously, the control assembly 130 can be coupled via a wired (e.g., Ethernet connection) or a wireless connection (e.g., via a WiFi™ network connection) to the server 150. In some embodiments, the control assembly 130 can be operatively coupled to the compute device 155 (e.g., a mobile compute device such as a smartphone) via a wired or wireless connection (e.g. Bluetooth® connection). In some implementations, the server 150, the memory 110, and/or the compute device 155 can include a database and be configured to provide information related to carrier materials, constituent substances included in a carrier material, information related to one or more constituent substances in a carrier material (e.g., known boiling points of a volatilizable constituent in an oil), standardized volumes associated with usage of a vaporizer, standardized quantities of volatilized components, a quantity of aerosols associated with the volatilization of a standard carrier material, vapor pressure, atmospheric pressure, and/or environmental or ambient temperatures associated with usage of a vaporizer at specific geographic locations, etc. In some implementations, the server 150, the memory 110, and/or the compute device 155 can include a database of materials providing information related to temperatures or temperature ranges at which the carrier material should be vaporized (e.g., as determined by a manufacturer of the vaporizer 100, a manufacturer of the carrier material, and/or a user of the vaporizer 100). The control assembly 130 can be configured to access the database and control the heating assembly 120 based, at least in part, on information provided in the database. Additionally, the server 150, the memory 110, and/or the compute device 155 can include a database of materials providing dosage information related to various carrier materials (e.g., amount of one or more constituent substances of included in a standard volume of a gaseous mixture including air and a volatized portion of a carrier material) (e.g., as determined by a manufacturer of the vaporizer 100, a manufacturer of the carrier material, and/or a user of the vaporizer 100). The control assembly 130 can be configured to receive the dosage information associated with a particular carrier material in the reservoir 104 from the database of the server 150, the memory 110, and/or the compute device 155 (e.g., in response to an identifier 119 or an indication of the identifier 119 being provided to the server 150, the memory 110, and/or the compute device 155).

The compute device 155 can each be any suitable hardware-based computing device and/or a multimedia device, such as, for example, a server, a desktop compute device, a smartphone, a tablet, a wearable device, a laptop and/or the like. In some instances, the compute device 155 can include a user interface including one or more control items and one or more display items configured to perform functions associated with communication with the vaporizer 100, remote control of the vaporizer 100, and/or display information associated with functioning or usage of the vaporizer 100.

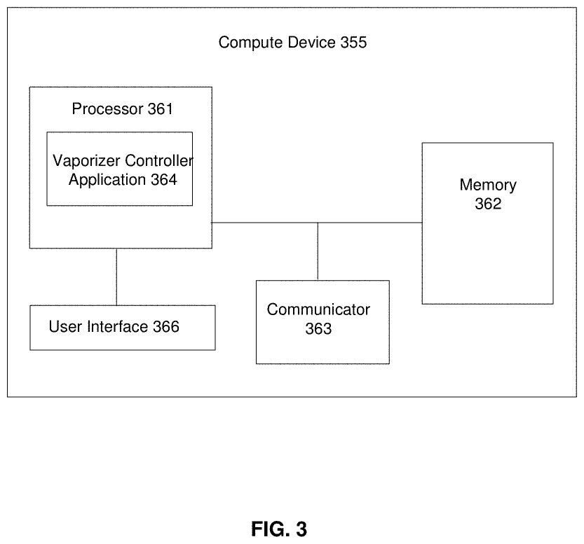

FIG. 3 is a block diagram of an example compute device 355 that can be included in a system configured for controlled dose delivery of one or more volatilized substances via a vaporizer, similar to the system 103 described above. The compute device 355 can be substantially similar in structure and/or function to the compute device 155 described with reference to the system 103 shown in FIG. 1. The compute device 355 can be a hardware-based computing device and/or a multimedia device, such as, for example, a server, a desktop compute device, a smartphone, a tablet, a wearable device, a laptop and/or the like. The compute device 355 includes a processor 361, a memory 362 (e.g., including data storage), and a communicator 363. The compute device 355 also includes a user interface 366 operatively coupled to the processor 361.

The memory 362 of the compute device 355 can be, for example, a random access memory (RAM), a memory buffer, a hard drive, a read-only memory (ROM), an erasable programmable read-only memory (EPROM), and/or the like. The memory 362 can store, for example, one or more software modules and/or code that can include instructions to cause the processor 361 to perform or instantiate or execute one or more processes, applications, interfaces, functions, and/or the like (e.g., the vaporizer controller application 364 described below and/or the user interface 366). In some embodiments, the memory 362 can include extendable storage units that can be added and used incrementally. In some implementations, the memory 362 can be a portable memory (for example, a flash drive, a portable hard disk, and/or the like) that can be operatively coupled to the processor 361. In other instances, the memory 362 can be remotely operatively coupled with the compute device 355. For example, a remote database server can serve as the memory 362 and be operatively coupled to the compute device 355.

The communicator 363 can be a hardware device operatively coupled to the processor 361 and the memory 362 and/or software stored in the memory 362 executable by the processor 361. The communicator 363 can be, for example, a network interface card (NIC), a Wi-Fi™ module, a Bluetooth® module and/or any other suitable wired and/or wireless communication device. Furthermore, the communicator 363 can include a switch, a router, a hub and/or any other network device. The communicator 363 can be configured to connect the compute device 361 to a communication network (such as the communication network 106 shown in FIG. 1). In some instances, the communicator 363 can be configured to connect to a communication network such as, for example, the Internet, an intranet, a local area network (LAN), a wide area network (WAN), a metropolitan area network (MAN), a worldwide interoperability for microwave access network (WiMAX®), an optical fiber (or fiber optic)-based network, a Bluetooth® network, a virtual network, and/or any combination thereof.

In some instances, the communicator 363 can facilitate receiving and/or transmitting a signal, a data set, information, a file and/or a set of files to and/or from a vaporizer (e.g. vaporizer 100) through a communication network (e.g., the communication network 106 in the system 103 of FIG. 1). In some embodiments, the communicator 363 can be configured to send and/or receive any suitable information associated with a functioning of the vaporizer in the system configured for controlled delivery of a dose of one or more volatilized substances using a vaporizer. For example, in some embodiments, the communicator 363 can be configured to send and/or receive information related to an identifier associated with a capsule portion of a vaporizer, an identity of a carrier material included in a reservoir of a capsule of the vaporizer, a dose of one or more constituent substances included in a portion of vaporized carrier material delivered from the vaporizer during a draw of the vaporizer, a predetermined threshold value associated with one or more constituent substances of a carrier material set by a user, and/or an identity associated with the capsule and or the pen portion of the vaporizer. In some embodiments, the communicator 363 can be configured to send and/or receive signals with adequate speed and/or timing such that information associated with a real-time functioning of the vaporizer can be exchanged between the vaporizer and the compute device 355 at a near-instantaneous rate. For example, the communicator 363 can be configured to facilitate near instantaneously sending and/or receiving, during a use of a vaporizer, information associated with indications related to one or more processes carried out at the vaporizer, such as, indications of an initiation of a draw at the vaporizer, indications related to a flow rate associated with a draw and/or a volume of air associated with a draw, indications related to a current supplied and/or ceased from being supplied to a heating element associated with the vaporizer, a signal causing a cessation of current to be supplied to a heating element, a signal indicating a strength associated with a draw (e.g., a measure of strength associated with a flow rate and/or duration of a draw), or the like. In some instances, the communicator 363 can facilitate receiving and/or sending signals related to synchronization of one or more processes executed at the compute device 355 with one or more processes executed at the vaporizer. The communicator 363 can be configured to communicate with a server (e.g., server 150 of the system 103 in FIG. 1) to send and/or receive information associated with a functioning of the vaporizer.

The processor 361 can be, for example, a hardware based integrated circuit (IC) or any other suitable processing device configured to run and/or execute a set of instructions or code. For example, the processor 361 can be a general purpose processor, a central processing unit (CPU), an accelerated processing unit (APU), an application specific integrated circuit (ASIC), a field programmable gate array (FPGA), a programmable logic array (PLA), a complex programmable logic device (CPLD), a programmable logic controller (PLC) and/or the like. The processor 361 can be operatively coupled to the memory 362 through a system bus (for example, address bus, data bus and/or control bus). The processor 211 is configured to execute a vaporizer controller application 364 with which a user can interact via the user interface 366 of the compute device 355. The vaporizer controller application 364 can be configured to identify (e.g., using one or more identifiers associated with a vaporizer such as a bar code, QR code, NFC signal etc.,) and connect via the communicator 363 to a control assembly of a vaporizer.

The processor 361 is configured to receive information from the vaporizer indicating an identifier associated with a capsule portion of a vaporizer, an identity of a carrier material included in a reservoir of a capsule of the vaporizer, a dose of one or more constituent substances included in a carrier material delivered during a draw of the vaporizer, a predetermined threshold value associated with one or more constituent substances of a carrier material set by a user, and/or the identity associated with the capsule and or the pen portion of the vaporizer. In some embodiments, the processor 361 can send and/or receive signals, via the communicator 363 and using the vaporizer controller application 364, with adequate speed and/or timing, such that information associated with a real-time functioning of the vaporizer can be exchanged between the vaporizer and the compute device at a near-instantaneous rate. For example, the processor 362 can be configured to near instantaneously send and/or receive, during a use of a vaporizer, information associated with indications related to one or more processes carried out at the vaporizer, such as, indications of an initiation of a draw at the vaporizer, indications related to a flow rate associated with a draw and/or a volume of air associated with a draw, indications related to a current supplied and/or ceased from being supplied to a heating element associated with the vaporizer, signal causing a cessation of current to be supplied to a heating element, a signal indicating a strength associated with a draw (e.g., a measure of strength associated with a flow rate and/or duration of a draw), or the like. In some instances, the processor 362 can receive and/or send signals related to synchronization of one or more processes executed at the compute device 355 with one or more processes executed at the vaporizer. For example, the processor 362 can be configured to synchronize an indication of a strength of draw provided to a user at the vaporizer (e.g., via indicators 112 associated with the vaporizer 100) with an indication of the strength of the same draw provided to the user at the compute device 355, for example via the user interface 366 associated with the compute device 355.

In some embodiments, the processor 362 can be configured to process (e.g., via the vaporizer controller application 364), one or more signals received from a vaporizer and present to the user, via the user interface 366, information associated with the signals in the form of feedback. For example, the processor 362 can receive information associated with one or more processes being carried out at the vaporizer and present the information via the user interface 366 using suitable graphical presentations. The information can include indications of an initiation of a draw at the vaporizer, indications related to a flow rate associated with a draw and/or a volume of air associated with a draw, indications related to a current supplied and/or ceased from being supplied to a heating element associated with the vaporizer, signal causing a cessation of current to be supplied to a heating element, a signal indicating a strength associated with a draw (e.g., a measure of strength associated with a flow rate and/or duration of a draw), or the like.

In some embodiments, the processor 362 can be configured to provide a user with a set of controls via the user interface 365 to set a threshold value that can be used to regulate a dose of one or more constituent substances included in a carrier material delivered during use of a vaporizer coupled to the compute device 355. The threshold value can be communicated to the vaporizer as a signal such that during a draw of the vaporizer the control assembly can receive and/or monitor a flow rate associated with the draw and based on the flow rate determine a volume of gaseous mixture delivered and/or a dose delivered of one or more volatilized constituent substances included in a volatilized carrier material. The control assembly can receive information associated with an amount of one or more of the constituent substances included in a standard volume of the gaseous mixture and based on the information determine a dose of each of the one or more constituent substances delivered to the user during the draw. The control assembly can compare the dose to the threshold value and as the dose nears or reaches the threshold the control assembly can cause a cessation of supply of current to the heating coil associated with the vaporizer such that there is no more continued delivery of the constituent substances.

FIG. 6A is a schematic of an example vaporizer 600. The vaporizer 600 can be the same or similar in structure and/or function to any of the vaporizers described herein, such as the vaporizer 100. For example, the vaporizer 600 can include a housing 610 substantially similar to the housing 110, a mouthpiece 602 substantially similar to the mouthpiece 102, and an indicator element 612 that is substantially similar to the indicator 112 described above with reference to the vaporizer 100.

The indicator 612 of the vaporizer 600 shown in FIG. 6A can be configured to include indicator features, for example, light-emitting diodes (LEDs). The indicator features may be configured to transmit light through a set of apertures in the housing of the vaporizer. The indicator features may be configured to indicate via one or more parameters associated with the light emitted (e.g., a wavelength of light emitted via the indicator features) such as a dose of one or more of the constituent substances included in the carrier material, a strength of a draw, and/or an indication of a relative degree of volume of gaseous mixture inhaled during a particular draw using the vaporizer 600 (e.g., an indication of how much of a portion of an intended draw or an amount of a substance included in the carrier material has been drawn from the mouthpiece 602 of the vaporizer 600).

As described previously with reference to the vaporizer 100, the control assembly of the vaporizer 600 can map the dose of one or more constituent substances delivered in a draw or a degree of volume of a draw to a LUT by generating the LUT as an array of varying wavelengths of light across a color map. The array of wavelengths in the LUT can be selected such that different levels of dose or different strength of draw or different degrees of volume correspond to a different portion of the LUT, respectively. The control assembly of the vaporizer 600 can be configured to cause the indicator features in indicator 612 to indicate a dose or degree of draw by emitting a light of a suitably modulated wavelength that corresponds to the dose or degree of draw according to the mapping of the LUT.

For example, a first indication (e.g., a blue wavelength) can be mapped to indicate a low dose compared, a weak draw, or a relatively small volume compared to a standard dose, a strength of a standard draw, or a standard volume of a gaseous mixture, respectively, during and/or after use of a vaporizer (e.g., during and/or after a user draws fluid through the mouthpiece 602). A second indication (e.g., an orange-red wavelength), as shown in the example illustration in FIG. 6A, can be mapped to indicate a large dose, a strong draw, or a relatively large volume compared to a standard dose, a strength of a standard draw, or a standard volume of a gaseous mixture, respectively, during use of a vaporizer. As illustrated the indicator 612 are shown to be emitting a light near an orange-red wavelength indicating a strong draw associated with a large volume of a gaseous mixture (e.g., vaporized carrier material) drawn through an opening in a mouthpiece of the vaporizer 600. In some instances, such a large draw can correspond to a large dose of one or more constituent substances included in the gaseous mixture.

Similar to the description of the vaporizer 100 above, the vaporizer 600 can be configured to allow a user to set a predetermined threshold dose associated with one or more of the constituent substances included in the carrier material. FIG. 6B illustrates a compute device 655 including a user interface 666 via a vaporizer controller application instantiated at the compute device 655. The vaporizer 600 can be operatively coupled to the compute device 655 which can be substantially similar to the compute device 155 and/or the compute device 355 described above. For example, the compute device 655 can include a vaporizer controller application that instantiates a user interface 666, which can be substantially similar to the user interface 366, described above. In some implementations, the user can set a predetermined threshold dose associated with one or more of the constituent substances included in the carrier material through the compute device 655 via the user interface 666.

In some implementations, as described previously, the user interface 666 instantiated at the compute device 655 can include a graphical presentation in the form of a colored wheel representing a range of values. In some implementations, the range of values can correspond to a range of doses. In some implementations, the range of values can correspond to a range of draw volumes. The wheel can include an indication 670, for example shown by a degree of fill with a mapped color scheme that is configured to move relative to a first point on the wheel and along the range of values, during a draw of the vaporizer 600. The movement of the indication 670 can be programmatically coupled to the draw such that the position of the indication at a given time during a draw is configured to indicate the cumulative dose of a substance drawn by the user, or a cumulative volume of gaseous mixture drawn by the user, at that time of the draw. Said in another way, in some instances, the graphical presentation of the wheel 667 can be dynamic such that the color wheel 667 is gradually filled with the color map during the draw with increasing filling being proportional to increasing volume drawn in a synchronous manner near instantaneously. The color wheel 667 can be configured to be filled incrementally in a clockwise sequence with increasing draw strength such that the point at which the fill is ceased indicates the final strength of the draw and the color at which the fill is ceased indicates the dose of one or more substances deliver during the draw using the vaporizer shown in FIG. 6A. The rate of movement of the indication 670 at a point in time during a draw can correspond to a flow rate associated with the draw indicating the draw strength at that point in time. Additionally, the rate of movement of the indication 670 may be lower when the heating element is in a pre-heat period of a heating profile compared to when the heating element is in a plateau period of the heating profile (even if the flow rate of air through the vaporizer during both periods is the same) since the dose of one or more constituent substances of the carrier material may be lower or at zero for a portion or the entirety of the pre-heat period compared to during a plateau period since the heating element has not yet heated the carrier material near the heating element to the intended vaporization temperature.

The processor of the compute device 655 can generate a LUT, for example, the LUT displayed by the color fill of the colored wheel 667 ranging from blue to orange to red, with blue color mapped to a portion of the wheel 667 indicating a low draw volume or low dose and orange-red mapped to a portion of the wheel 667 indicating a relatively large draw volume or large dose of a carrier material including one or more constituent substances. In some instances, the processor can receive information associated with the LUT from the vaporizer 600 to be used to indicate the draw strength and/or the dose of a set of constituent substances included in a carrier material associated with the vaporizer 600.

As shown, the wheel 667 indicates via the color and position of the indication 670 (also shown by the fill of the color wheel), a volume associated with a draw (also referred to as a “pull”) through a mouthpiece 602 of the vaporizer 600 and/or a dose of one or more constituent substances inhaled during an example draw using the vaporizer 600 shown in FIG. 6A. In some implementations, the wheel 667 can be configured to use a similar LUT as the one used by the vaporizer 600 to cause the indicator features of indicator 612 to emit light of a specified wavelength, and the indicator 612 and the wheel 667 can be configured to indicate, in a synchronized manner, a volume associated with a draw and/or a dose of one or more constituent substances inhaled during the draw. Said in another way, in some implementations, the vaporizer 600 and the user interface 666 can be configured such that the wavelength of light emitted by the indicators 612, at a given time during a draw of the vaporizer 600, to indicate a cumulative volume associated with the draw and/or a dose of one or more constituent substances inhaled during the draw, matches a wavelength of color at the position of the indication 670 on the wheel 667 at that given time during the draw. As shown the color at the point where fill of the color wheel 667 is stopped to indicate a dose of constituent substances can be configured to match the wavelength of the light emitted by the indicator 612 in the vaporizer. In some embodiments, the indication via the indicator 612 and the dynamic graphical presentation via the color wheel 667 can be synchronous and near instantaneous with usage of the vaporizer 600, such that immediate real-time feedback can be used to educate (e.g., coach) a user with respect to the strength and/or duration of a draw needed to reach a desired dose of one or more substances.

In use, as an example implementation, the processor of the compute device 655 can receive a signal from the vaporizer 600, during a particular use of the vaporizer 600, indicating one or more parameters associated with the use. The one or more parameters can include a near instantaneous flow rate associated with a draw, a volume of gaseous mixture associated with the draw, and/or a dose of one or more constituent substances identified to be included in the carrier material and drawn from the mouthpiece opening of the vaporizer 600. The processor can be configured to map the received signal to the LUT to determine a representative color to indicate the dose of one or more substances included in the carrier material. The processor can also be configured to determine a relative measure of flow rate associated with the draw with reference to a standard draw or a predetermined draw of a gaseous mixture. For example, the processor can determine a percentage or proportionality value of the strength of the particular draw with reference to the standard draw or the predetermined draw. The processor is configured to then generate the display of the color wheel 667 such that the fill level indicates the relative measure of strength associated with the particular draw and the color at the point where the fill is ceased indicates the dose of one or more substances delivered during the draw. As shown in the example in FIG. 6B, the color wheel 667 indicates a strong draw (e.g., approximately 80% the strength of a standard draw or a predetermined draw) and a large dose (e.g., 10.5 mg) of the carrier material drawn from the vaporizer.

The user interface 666, in some implementations, can include separate dose indicators identifying the particular amount of each substance drawn from the mouthpiece opening. The chart 668 shown in FIG. 6B is configured to correspond to the indication via the color wheel 667 and display a dose associated with four example constituent substances included in the carrier material, namely THC with a dose of 5.02 mg, CBD with a dose of 0.3 mg, CBC with a dose of 0.0 mg, and terpenes with a dose of 0.002 mg.

As shown in FIG. 6B, the user interface 666 can provide the user with controls 669 that can be used to set thresholds (e.g., a first, second and third threshold mapping to a small, medium and a large draw and/or dose, indicated by the respective colors of the controls and the location of the controls alongside the color wheel indicating the range of strength of draw). Other embodiments of a user interface can be used to set any number of thresholds using suitably small or large increments in values associated with small or large increments in draw volumes and/or doses. As shown, in an example instance, the third threshold can be selected by a user (e.g., by clicking the third control 669 or by touching the third control via a touchscreen display) indicated by the circle around the control. The selection of the threshold can be communicated to the vaporizer 600 such that as indicated the delivery of volatilized substance during the draw can be ceased when the draw strength reaches the threshold level (as indicated by the unfilled grayed portion of the color wheel 667). In some embodiments, the threshold can also be set via an optional dose control button included in a vaporizer (e.g., dose control button 118 of vaporizer 100 in FIG. 2). For example, in some instances, a number of presses of the dose control button can be received and mapped to a threshold level (e.g., one press mapping to a threshold at a small dose of one or more constituents included in a carrier material, two presses mapping to a medium dose of one or more constituents included in a carrier material, and three presses mapping to a threshold at a large dose of one or more constituents included in a carrier material). In some embodiments, the selection of the thresholds or controls 669 can correspond to a user selection of a heating profile (e.g., a predetermined temperature adjustment curve over an inhalation period) (also referred to as a vapor style). For example, the first control 669 can correspond to a first heating profile, the second control 669 can correspond to a second heating profile, and the third control 669 can correspond to a third heating profile. Each of the heating profiles can be based, at least in part, on the carrier material to be used with the vaporizer 600. In some embodiments, selection of the first heating profile may cause the carrier material to be vaporized at a lower temperature, resulting in a stronger, fuller, or otherwise improved flavor but a lower dosage of delivered constituent substance(s) per draw, while selection of the third heating profile may cause the carrier material to be vaporized at a higher temperature, resulting in a weaker or otherwise less intense flavor but a higher dosage of delivered constituent substance(s) per draw. The second heating profile may result in stronger flavor and a higher delivered dosage per draw than the third heating profile and weaker flavor and lower delivered dosage than the first heating profile.

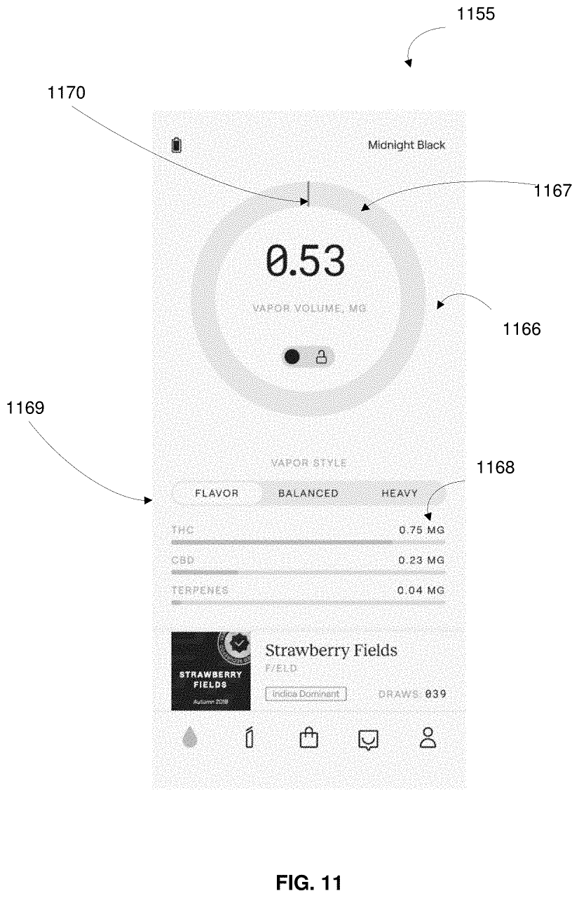

FIG. 11 illustrates a compute device 1155 including a user interface 1166 via a vaporizer controller application instantiated at the compute device 1155. A vaporizer, such as any of the vaporizers described herein, can be operatively coupled to the compute device 1155 which can be substantially similar to the compute device 155, the compute device 355, and/or the compute device 655 described above. For example, the compute device 1155 can include a vaporizer controller application that instantiates a user interface 1166, which can be substantially similar to the user interface 666, described above. Additionally, the user interface 1166 can include chart 1168, wheel 1167, and indication 1170, which can be substantially similar to the chart 668, wheel 1167, and indication 1170 described above.

In some implementations, the user can select a particular style of vapor delivery associated with one or more of the constituent substances included in the carrier material through the compute device 1155 via the user interface 1166. For example, as shown in FIG. 11, the user interface includes three selection buttons 1169 (e.g., which may be presented as a slider) allowing the user to select one of a first vapor style (represented as “flavor”), a second vapor style (represented as “balanced”), and a third vapor style (represented as “heavy”). The first vapor style 1169 (also referred to herein as a first control) can correspond to a first heating profile, the second vapor style 1169 (also referred to herein as a second control) can correspond to a second heating profile, and the third vapor style 1169 (also referred to herein as a third control) can correspond to a third heating profile. Each of the heating profiles can be based, at least in part, on the carrier material to be used with the vaporizer associated with the compute device 1155. In some embodiments, selection of the first heating profile may cause the carrier material to be vaporized at a lower temperature, resulting in a stronger, fuller, or otherwise improved flavor but a lower dosage of delivered constituent substance(s) per draw, while selection of the third heating profile may cause the carrier material to be vaporized at a higher temperature, resulting in a weaker or otherwise less intense flavor but a higher dosage of delivered constituent substance(s) per draw. The second heating profile may result in stronger flavor and a higher delivered dosage per draw than the third heating profile and weaker flavor and lower delivered dosage than the first heating profile.

In some embodiments, any of the vaporizers described herein can include one or more indicators on the device body configured to indicate an inhaled dosage to the user during inhalation. For example, a vaporizer can include a visual indicator (e.g., including one or more LEDs) that is visible to the user during inhalation through a mouthpiece opening of the vaporizer and/or a vibration motor configured to provide haptic feedback to the user such that the user can feel the vibration through a hand of the user holding the vaporizer during inhalation. In some implementations, the visual indicator and/or vibration motor can be activated when the user has inhaled a first dosage amount (e.g., 2 mg). The visual indicator and/or vibration motor can be reactivated at particular dosage intervals beyond the first dosage amount (e.g., every half milligram or milligram) as the user continues to apply suction to the mouthpiece.

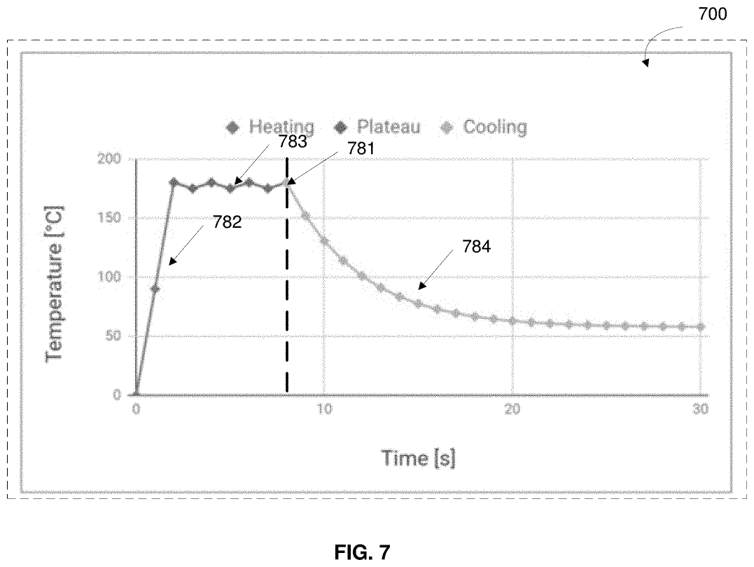

As described above, the vaporizers described herein (e.g., vaporizer 100, 600) can be configured to control the delivery of the dose during use such that when a measured dose of the one or more constituent substances reaches or nears the predetermined threshold, the vaporizer can be configured to cease the continued delivery of the one or more constituent substances by ceasing supply of current to the heating element. This is shown by an example heating profile 700 of a temperature associated with a heating coil of a vaporizer in FIG. 7.

FIG. 7 illustrates an example heating profile 700 associated with a single draw during usage of a vaporizer, according to an embodiment. The heating profile 700 is a representative example of a heating profile of a heating element of a heating assembly that can be generated by a control assembly that is the same or similar to any of the control assemblies described herein, such as the control assembly 130. As shown in FIG. 7, the heating profile 700 can have a ramp-up or heating phase 782 in which the temperature of the heating element rises. After the heating phase 782, the heating profile 700 can include a set-point phase or a plateau phase 783 (also referred to as a body phase) in which the temperature of the heating element is maintained at a temperature or within a range of temperatures. The plateau phase 783 can be configured to have a duration associated with a duration of an inhalation by a user. The plateau phase 783 can in some instances be terminated based on a threshold crossing event where a control assembly can determine, based on a volume of gaseous mixture delivered during that draw and/or a dose of one or more substances delivered during the draw, and a predetermined threshold value set by the user, to cease continued delivery of the volatilized substance by terminating the plateau phase 783 and initiating the amp-down or cooling phase 784. In the heating profile 700 the temperature is shown to ramp up and plateau following heating of the coil during the heating and plateau phases and to drop during the cooling phase following a point 781 indicated by the dashed line. The point 781 can correspond, for example, to a point in time when a dose delivered was identified (e.g., by a processor of the vaporizer or a processor of a compute device coupled to the vaporizer) to cross a threshold and, in response, a delivery of current to the heating element was discontinued. During the ramp-down or a cooling phase 784 the temperature of the heating element can passively cool to a temperature corresponding to the ambient temperature.

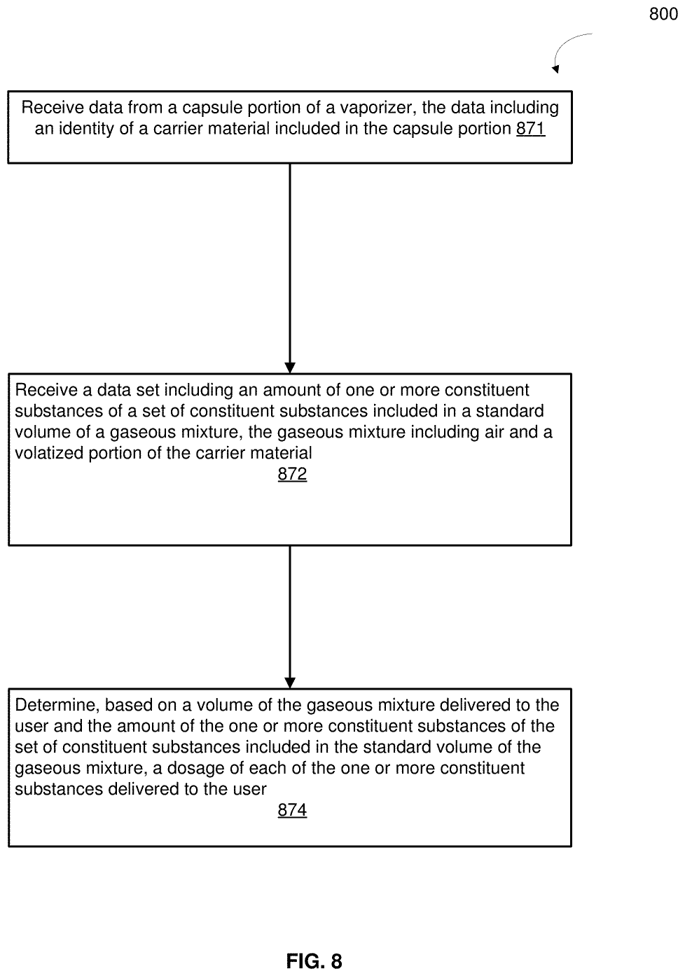

FIG. 8 is a flowchart describing an example method 800 of determining a dose of one or more constituent substances delivered to a user of a vaporizer, according to an embodiment. A processor implementing the method 800 receives data, at 871, from a capsule portion of a vaporizer, the data including an identity of a carrier material included in the capsule portion. In some implementations, the data can be received from a tracking component associated with the capsule assembly. The tracking component may be programmed (e.g., by a manufacturer of the vaporizer or the capsule portion) to include information related to the specific carrier material disposed in the reservoir associated with the capsule assembly. In some implementations, the tracking component may provide information related to an identity of the specific carrier material and/or the constituent components of the specific carrier material. For example, the tracking component may provide the identity of volatilizable components included in the carrier material such as phytocannabinoids, terpenes, etc. In some embodiments, the method 800 optionally includes receiving data from a compute device and/or a server related to the characteristics of the carrier material and/or heating instructions (e.g., instead of or in addition to receiving data from the capsule portion or the tracking portion).

At 872, the processor receives a data set including an amount of one or more constituent substances of a set of constituent substances included in a standard volume of a gaseous mixture, the gaseous mixture including air and a volatized portion of the carrier material. In some embodiments, the processor can receive this information from a compute device and/or a remote server hosted by a third party organization, as described previously. In some implementations, the processor may receive via the server 150 a data set including a standard amount (e.g., a certified standard amount) of the one or more constituent substances included in a known carrier material delivered during a draw of a standard volume of a gaseous mixture generated from vaporizing the known carrier material under a standard regime of usage of a vaporizer (e.g., a standard draw using a standard vaporizer). The length of the standard draw can be, for example, 3 second or 4 seconds.

At 873, the processor determines, based on a volume of the gaseous mixture delivered to the user and the amount of the one or more constituent substances of the set of constituent substances included in the standard volume of the gaseous mixture, a dose of each of the one or more constituent substances delivered to the user. In some implementations, the processor can receive, from a flow sensor associated with the vaporizer, information related to a duration of flow and/or flow rate of air drawn by a user of the vaporizer. The processor can then calculate a volume of the gaseous mixture delivered to the user during a particular draw, the gaseous mixture including air and a volatized portion of the carrier material. In some implementations, the processor can receive, from a pressure sensor associated with the vaporizer, information related to a change in pressure associated with fluids (e.g., air, gaseous mixture including aerosols from volatilized portion of the carrier material included in the vaporizer, etc.,) held in one or more portions of the vaporizer (e.g., the fluidic channels, or the one or more chambers included in the vaporizer). The change in pressure may be associated with a draw of air by a user of the vaporizer. The processor can calculate a volume of the gaseous mixture delivered to the user during a particular draw, the gaseous mixture including air and a volatized portion of the carrier material. The processor can use the change in pressure data over time to determine (e.g., calculate) the flow rate through the vaporizer (e.g., the fluid channels and chambers) and out of the opening in the mouthpiece. The flow rate can then be used to calculate the volume of the gaseous mixture delivered to the user during a particular draw.

The processor can identify a relationship (e.g., a ratio) between the standard volume of the gaseous mixture and the volume of the gaseous mixture delivered to the user during the particular draw. Based on the volume of the gaseous mixture delivered to the user and the amount of the one or more constituent substances of the set of constituent substances included in the standard volume of the gaseous mixture, and the relationship between the standard volume of the gaseous mixture and the volume of the gaseous mixture delivered to the user during the particular draw, the processor can determine a dose of each of the one or more constituent substances delivered to the user during the particular draw. In some implementations, the processor can send the determined dose information to one or more indicators and/or a compute device to be displayed to the user as feedback.

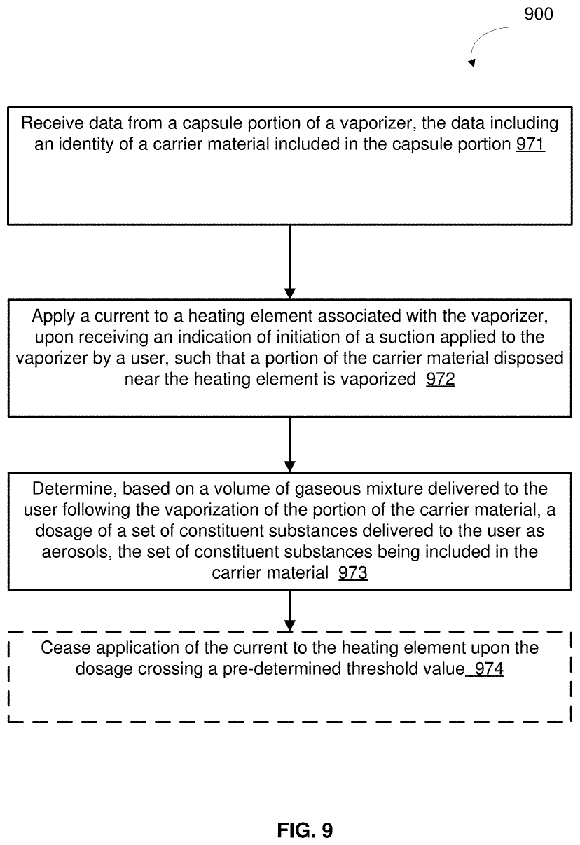

FIG. 9 is a flowchart describing an example method 900 of controlling a dose of one or more constituent substances delivered to a user of a vaporizer, such as any of the vaporizers described herein, according to an embodiment. A processor (e.g., of a pen portion such as any of the pen portions described herein) implementing the method 900 receives data, at 971, from a capsule portion (e.g., such as any of the capsule portions described herein) of a vaporizer, the data including an identity of a carrier material included in the capsule portion. In some implementations, the data can be received from a tracking component associated with the capsule assembly. The tracking component may be programmed (e.g., by a manufacturer of the vaporizer or the capsule portion) to include information related to the specific carrier material disposed in the reservoir associated with the capsule assembly. In some implementations, the tracking component may provide information related to an identity of the specific carrier material and/or the constituent components of the specific carrier material. For example, the tracking component may provide the identity of volatilizable components included in the carrier material such as phytocannabinoids, terpenes, etc. In some embodiments, the method 900 optionally includes receiving data from a compute device and/or a server related to the characteristics of the carrier material and/or heating instructions.

In some embodiments, a specific heating profile and/or dose per standard volume can be stored in the tracking component and provided to the processor when the capsule portion is operatively coupled to the pen portion. In some embodiments, the processor can be configured to request a specific heating profile and/or dose per standard volume associated with the carrier material within the capsule portion from a compute device and/or a remote server (e.g., via a compute device) based on the identification data received from the capsule portion. In some embodiments, the processor can be configured to send one or more signals to the tracking component (e.g., to an integrated circuit of the tracking component) of the capsule portion such that a specific heating profile and/or dose per standard volume is stored in the tracking component. For example, in some embodiments, when a capsule portion is initially coupled with a pen portion (and has not been previously coupled with a pen portion since being filled with carrier material), a processor of the pen portion can retrieve information (e.g., a specific heating profile and/or dose per standard volume) specific to the carrier material and store the information (e.g., write or program the information) on a tracking component of the capsule portion. For any subsequent uses of the capsule portion, the processor of the pen portion (or a processor of another pen portion to which the capsule portion may be coupled), can retrieve the information specific to the carrier material in the capsule portion (the specific heating profile and/or dose per standard volume) from the tracking component of the capsule portion, rather than from a compute device and/or a remote server.

At 972, the processor applies a current to a heating element associated with the vaporizer, upon receiving an indication of initiation of a suction applied to the vaporizer by a user, such that a portion of the carrier material disposed near the heating element is vaporized. In some implementations, a user may draw air through the mouthpiece of the vaporizer by applying the user's mouth to the mouthpiece and applying negative pressure to the mouthpiece opening (e.g., by sucking). In implementations including a pressure sensor in communication with the control assembly, the negative pressure may trigger the pressure sensor. In response to receiving indication of negative pressure from the pressure sensor, the processor may actuate a heater control circuitry associated with the control assembly including the processor such that a current is passed through a heating element such as a coil. The coil can be heated to a particular temperature based on a predetermined heating profile (e.g., heating profile 700). The pressure sensor in combination with the processor may be configured to determine that flow is occurring through the mouthpiece opening and the rate of the flow of the gaseous mixture being delivered (e.g., based on the change in pressure over time). In some embodiments, the current provided to the coil may be based, at least in part, on the flow rate and/or duration of flow as determined based on the change in pressure sensed by the pressure sensor. Alternatively, in implementations including an activation button (not shown) in communication with the control assembly, the user may actuate the activation button such that the control assembly in response to receiving an actuation signal from the activation button, may actuate heater control circuitry of the control assembly such that a current is passed through the coil and the coil is heated to a particular temperature.

At 973, the processor determines, based on a volume of gaseous mixture delivered to the user following the vaporization of the portion of the carrier material, a dose of a set of constituent substances delivered to the user as aerosols, the set of constituent substances being included in the carrier material. As described with reference to method 800, the processor can use information associated with a standard volume of the gaseous mixture to calculate the dose of the set of constituent substances delivered to the user. For example, as described previously, the processor can receive a data set including an amount of one or more constituent substances of a set of constituent substances included in a standard or predetermined volume of a gaseous mixture, the gaseous mixture including air and a volatized portion of the carrier material. In some embodiments, the processor can receive this information (e.g., in response to requesting the information based on the data received from the capsule portion) from a compute device and/or a remote server, as described previously. In some implementations, the processor may receive via a server coupled to the vaporizer a data set including a standard amount (e.g., a certified standard amount) of the one or more constituent substances included in a known carrier material delivered during a draw of a standard volume of a gaseous mixture generated from vaporizing the known carrier material under a standard regime of usage of a vaporizer (e.g., a standard draw using a standard vaporizer). Based on a volume of the gaseous mixture delivered to the user and the amount of the one or more constituent substances of the set of constituent substances included in the standard volume of the gaseous mixture, and a relationship (e.g., a ratio) between the standard volume of the gaseous mixture and the volume of the gaseous mixture delivered to the user during the particular draw, the processor can determine the dose of each of the one or more constituent substances delivered to the user during the particular draw.

At 974, the processor optionally ceases application of the current to the heating element upon the dose reaching or crossing a pre-determined threshold value. For example, the process can determine and/or receiving an indication that the dose crossed a pre-determined threshold value. In some implementations, a user can set a predetermined threshold dose associated with one or more of the constituent substances included in the carrier material and operatively control the delivery of the dose during use such that when a measured dose of the one or more constituent substances reaches, passes, or nears the predetermined threshold, the processor can cause the vaporizer to cease the continued delivery of the one or more constituent substances by ceasing the supply of current to the heating element. Following the ceasing of the application of current, the heating element may be allowed to cool down passively to ambient temperature. In some embodiments, further drawing air through the vaporizer by a user after current has been ceased to the heating element can reduce the temperature of the heating element to or toward an ambient temperature due to the ambient air being pulled over or near the heating element. The processor can take into account the desired threshold dose, the heating and/or cooling properties of the heating element, the ramp up period (e.g., a pre-heating period) or a portion of the ramp up period of the temperature of the heating element and/or the portion of the carrier material disposed near the heating element (during which time some or all of the constituents of the carrier material may not be evaporating and thus dosing of those constituents has not yet started), and any residual vaporization that is expected during an initial portion of the ramp down following the ceasing of application of current to determine the point of ceasing the application of current to the heating element. For example, in some embodiments, the calculation of the volume of the gaseous mixture drawn through and/or from the vaporizer may be based on a calculation or measurement of the amount of gaseous mixture drawn through and/or from the vaporizer after the heating element and/or the portion of the carrier material disposed near the heating element has reached a threshold percentage of the intended vaporization temperature or temperature range, such as, for example, 50%, 60%, 70%, 80%, or 90%. In some instances where a threshold crossing event fails to occur (e.g., the draw of a vaporizer does not induce delivery of one or more constituent substances of a carrier material to a threshold value), the processor may apply a predetermined heating profile to determine a point of termination of the application of current to the heating element or terminate the application of current following termination of the draw by the user.

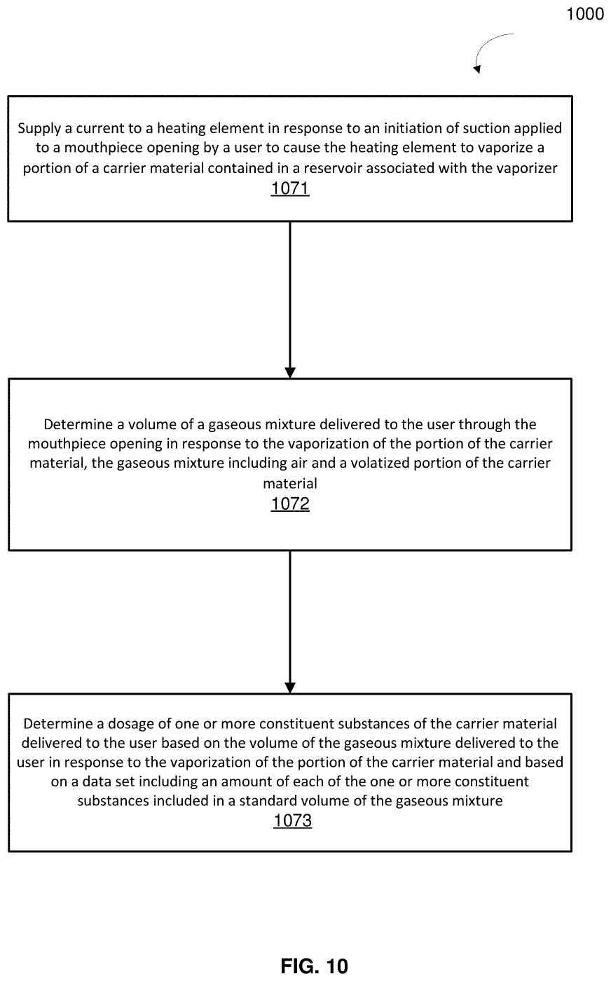

FIG. 10 is a flowchart describing an example method 1000 of controlling a dose of one or more constituent substances delivered to a user of a vaporizer, such as any of the vaporizers described herein, according to an embodiment. The method 1000 includes supplying, at 1071, a current to a heating element in response to an initiation of suction applied to a mouthpiece opening by a user to cause the heating element to vaporize a portion of a carrier material contained in a reservoir associated with the vaporizer. A volume of a gaseous mixture delivered to the user through the mouthpiece opening in response to the vaporization of the portion of the carrier material can be determined, at 1072. The gaseous mixture including air and a volatized portion of the carrier material. A dosage of one or more constituent substances of the carrier material delivered to the user based on the volume of the gaseous mixture delivered to the user in response to the vaporization of the portion of the carrier material and based on a data set including an amount of each of the one or more constituent substances included in a standard volume of the gaseous mixture can be determined, at 1073. In some embodiments, the standard volume of the gaseous mixture is a volume associated with a standard duration of suction applied to the mouthpiece opening.

In some embodiments, the method 1000 includes ceasing application of the current to the heating element upon the dosage of at least one of the one or more constituent substances crossing a pre-determined threshold value. In some embodiments, the method 100 includes receiving the data set including the amount of each of the one or more constituent substances included in the standard volume of the gaseous mixture from a remote compute device configured to communicate with the control assembly in response to receiving an indication of an identifier associated with the carrier material. In some embodiments, the method includes providing an indication of the dosage of the one or more constituent substances of the carrier material delivered to the user via an indicator including a vibration motor configured to provide haptic feedback to the user.

Some embodiments described herein relate to a computer storage product with a non-transitory computer-readable medium (also can be referred to as a non-transitory processor-readable medium) having instructions or computer code thereon for performing various computer-implemented operations. The computer-readable medium (or processor-readable medium) is non-transitory in the sense that it does not include transitory propagating signals per se (e.g., a propagating electromagnetic wave carrying information on a transmission medium such as space or a cable). The media and computer code (also can be referred to as code) may be those designed and constructed for the specific purpose or purposes. Examples of non-transitory computer-readable media include, but are not limited to, magnetic storage media such as hard disks, floppy disks, and magnetic tape; optical storage media such as Compact Disc/Digital Video Discs (CD/DVDs), Compact Disc-Read Only Memories (CD-ROMs), and holographic devices; magneto-optical storage media such as optical disks; carrier wave signal processing modules; and hardware devices that are specially configured to store and execute program code, such as Application-Specific Integrated Circuits (ASICs), Programmable Logic Devices (PLDs), Read-Only Memory (ROM) and Random-Access Memory (RAM) devices. Other embodiments described herein relate to a computer program product, which can include, for example, the instructions and/or computer code discussed herein.

Some embodiments and/or methods described herein can be performed by software (executed on hardware), hardware, or a combination thereof. Hardware modules may include, for example, a general-purpose processor, a field programmable gate array (FPGA), and/or an application specific integrated circuit (ASIC). Software modules (executed on hardware) can be expressed in a variety of software languages (e.g., computer code), including C, C++, Java™, Ruby, Visual Basic™, and/or other object-oriented, procedural, or other programming language and development tools. Examples of computer code include, but are not limited to, micro-code or micro-instructions, machine instructions, such as produced by a compiler, code used to produce a web service, and files containing higher-level instructions that are executed by a computer using an interpreter. For example, embodiments may be implemented using imperative programming languages (e.g., C, Fortran, etc.), functional programming languages (Haskell, Erlang, etc.), logical programming languages (e.g., Prolog), object-oriented programming languages (e.g., Java, C++, etc.) or other suitable programming languages and/or development tools. Additional examples of computer code include, but are not limited to, control signals, encrypted code, and compressed code.

Various concepts may be embodied as one or more methods, of which at least one example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments. Put differently, it is to be understood that such features may not necessarily be limited to a particular order of execution, but rather, any number of threads, processes, services, servers, and/or the like that may execute serially, asynchronously, concurrently, in parallel, simultaneously, synchronously, and/or the like in a manner consistent with the disclosure. As such, some of these features may be mutually contradictory, in that they cannot be simultaneously present in a single embodiment. Similarly, some features are applicable to one aspect of the innovations, and inapplicable to others.

In addition, the disclosure may include other innovations not presently described. Applicant reserves all rights in such innovations, including the right to embodiment such innovations, file additional applications, continuations, continuations-in-part, divisionals, and/or the like thereof. As such, it should be understood that advantages, embodiments, examples, functional, features, logical, operational, organizational, structural, topological, and/or other aspects of the disclosure are not to be considered limitations on the disclosure as defined by the embodiments or limitations on equivalents to the embodiments. Depending on the particular desires and/or characteristics of an individual and/or enterprise user, database configuration and/or relational model, data type, data transmission and/or network framework, syntax structure, and/or the like, various embodiments of the technology disclosed herein may be implemented in a manner that enables a great deal of flexibility and customization as described herein.

All definitions, as defined and used herein, should be understood to control over dictionary definitions, definitions in documents incorporated by reference, and/or ordinary meanings of the defined terms.

As used herein, in particular embodiments, the terms “about” or “approximately” when preceding a numerical value indicates the value plus or minus a range of 10%. Where a range of values is provided, it is understood that each intervening value, to the tenth of the unit of the lower limit unless the context clearly dictates otherwise, between the upper and lower limit of that range and any other stated or intervening value in that stated range is encompassed within the disclosure. That the upper and lower limits of these smaller ranges can independently be included in the smaller ranges is also encompassed within the disclosure, subject to any specifically excluded limit in the stated range. Where the stated range includes one or both of the limits, ranges excluding either or both of those included limits are also included in the disclosure.

The indefinite articles “a” and “an,” as used herein in the specification and in the embodiments, unless clearly indicated to the contrary, should be understood to mean “at least one.”

The phrase “and/or,” as used herein in the specification and in the embodiments, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the embodiments, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the embodiments, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of” or “exactly one of.” “Consisting essentially of,” when used in the embodiments, shall have its ordinary meaning as used in the field of patent law.

As used herein in the specification and in the embodiments, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the embodiments, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

While specific embodiments of the present disclosure have been outlined above, many alternatives, modifications, and variations will be apparent to those skilled in the art. Accordingly, the embodiments set forth herein are intended to be illustrative, not limiting. Various changes may be made without departing from the spirit and scope of the disclosure. Where methods and steps described above indicate certain events occurring in a certain order, those of ordinary skill in the art having the benefit of this disclosure would recognize that the ordering of certain steps may be modified and such modification are in accordance with the variations of the invention. Additionally, certain of the steps may be performed concurrently in a parallel process when possible, as well as performed sequentially as described above. The embodiments have been particularly shown and described, but it will be understood that various changes in form and details may be made.

1. A system, comprising:
a mouthpiece defining a mouthpiece opening;
a reservoir configured to contain carrier material including one or more constituent substances;
a heating assembly including a heating element configured to apply heat to the carrier material to vaporize the carrier material; and
a control assembly configured to:
supplying a current to the heating element in response to an initiation of suction applied to the mouthpiece opening by a user to cause the heating element to vaporize a portion of the carrier material,
determine a volume of a gaseous mixture delivered to the user in response to the vaporization of the portion of the carrier material, the gaseous mixture including air and a volatized portion of the carrier material, and
determine a dosage of each of the one or more constituent substances delivered to the user based on the volume of the gaseous mixture delivered to the user in response to the vaporization of the portion of the carrier material and based on a data set including an amount of each of the one or more constituent substances included in a standard volume of the gaseous mixture.

2. The system of claim 1, wherein the control assembly is configured to determine the volume of the gaseous mixture delivered to the user in response to the vaporization of the portion of the carrier material based, at least in part, on a duration of the suction applied to the mouthpiece opening by the user.

3. The system of claim 1, wherein the control assembly is configured to receive the data set including the amount of each of the one or more constituent substances include in the standard volume of the gaseous mixture from a tracking component disposed in a capsule portion operably coupleable to the control assembly.

4. The system of claim 1, wherein the control assembly is configured to receive the data set including the amount of each of the one or more constituent substances included in the standard volume of the gaseous mixture from a remote compute device configured to communicate with the control assembly in response to receiving an indication of an identifier associated with the carrier material.

5. The system of claim 1, wherein the standard volume of the gaseous mixture is a volume associated with a standard duration of suction applied to the mouthpiece opening.

6. The system of claim 1, further comprising an indicator, wherein the control assembly is configured to control the indicator to represent the dosage of each of the one or more constituent substances delivered to the user.

7. The system of claim 6, wherein the indicator includes a vibration motor configured to provide haptic feedback to the user.

8. The system of claim 6, wherein the indicator includes a visual display configured to be viewed by the user during an application of suction to the mouthpiece opening.

9. The system of claim 1, wherein the control assembly is configured to send the dosage of each of the one or more constituent substances delivered to the user such that the dosage of each of the one or more constituent substances delivered to the user can be displayed on the remote compute device.

10. The system of claim 1, wherein the control assembly is configured to cease application of the current to the heating element upon the dosage of at least one of the one or more constituent substances crossing a pre-determined threshold value.

11. A method, comprising:
supplying a current to a heating element in response to an initiation of suction applied to a mouthpiece opening by a user to cause the heating element to vaporize a portion of a carrier material contained in a reservoir associated with the vaporizer;
determining a volume of a gaseous mixture delivered to the user through the mouthpiece opening in response to the vaporization of the portion of the carrier material, the gaseous mixture including air and a volatized portion of the carrier material; and
determining a dosage of one or more constituent substances of the carrier material delivered to the user based on the volume of the gaseous mixture delivered to the user in response to the vaporization of the portion of the carrier material and based on a data set including an amount of each of the one or more constituent substances included in a standard volume of the gaseous mixture.

12. The system of claim 11, further comprising ceasing application of the current to the heating element upon the dosage of at least one of the one or more constituent substances crossing a pre-determined threshold value.

13. The system of claim 11, further comprising receiving the data set including the amount of each of the one or more constituent substances included in the standard volume of the gaseous mixture from a remote compute device configured to communicate with the control assembly in response to receiving an indication of an identifier associated with the carrier material.

14. The system of claim 11, wherein the standard volume of the gaseous mixture is a volume associated with a standard duration of suction applied to the mouthpiece opening.

15. The system of claim 11, further comprising providing an indication of the dosage of the one or more constituent substances of the carrier material delivered to the user via an indicator including a vibration motor configured to provide haptic feedback to the user.

16. A method, comprising:
receiving data including an identity of a carrier material disposed in a reservoir associated with a vaporizer,
receiving a data set including an amount of one or more constituent substances of a set of constituent substances included in a standard volume of a gaseous mixture, the gaseous mixture including air and a volatized portion of the carrier material; and
determining, based on a volume of gaseous mixture delivered to the user and the amount of the one or more constituent substances of the set of constituent substances included in the standard volume of the gaseous mixture, a dosage of each of the one or more constituent substances delivered to the user.

17. The method of claim 16, wherein the data including the identity of the carrier material is received from a capsule portion of the vaporizer and the data includes the carrier material included in the capsule portion.

18. The method of claim 16, wherein the data set including the amount of the one or more constituent substances of the set of constituent substances included in the standard volume of a gaseous mixture is received from a remote compute device.

19. The system of claim 16, wherein the standard volume of the gaseous mixture is a volume associated with a standard duration of suction applied to a mouthpiece opening of the vaporizer.

20. The system of claim 16, further comprising providing an indication of the dosage of one or more of the constituent substances of the carrier material delivered to the user via an indicator including a vibration motor configured to provide haptic feedback to the user.
